(19) United States
(12) Patent Application Publication
(10) Pub. No.: US 2024/0252548 A1
(43) Pub. Date: Aug. 1, 2024

(54) ADIPOSE COMPOSITIONS AND METHODS OF USE THEREOF

(71) Applicant: BRITECYTE INC., Frederick, MD (US)

(72) Inventor: Alla DANILKOVITCH, Columbia, MD (US)

(21) Appl. No.: 18/578,959

(22) PCT Filed: Jul. 13, 2022
(86) PCT No.: PCT/US2022/036982

§ 371 Date: Jan. 12, 2024

Related U.S. Application Data
(60) Provisional application No. 63/221,248, filed on Jul. 13, 2021.

(51) Int. Cl. A61K 35/35; A61K 9/00; A61K 35/12; A61K 35/28; A61P 1/16; A61P 3/06

(52) U.S. Cl. CPC A61K 35/35; A61K 9/0019; A61K 35/28; A61P 1/16; A61P 3/06; A61K 2035/122

(57) ABSTRACT

Disclosed are adipose matrices comprising devitalized, or vitalized, and partially delipidized adipose tissue. Disclosed are compositions comprising an adipose matrix comprising devitalized, or vitalized, and partially delipidized adipose tissue. Disclosed are compositions comprising an adipose matrix comprising devitalized, or vitalized, and partially delipidized adipose tissue and MSCs. Disclosed are methods of making any of the disclosed adipose matrices or compositions. Disclosed are methods of treating a subject having a metabolic liver disease, metabolic disease, ischemic wound, or inflammatory disease comprising administering to the subject a therapeutically effective amount of a composition disclosed herein. Disclosed are methods of decreasing cholesterol in a subject comprising administering to the subject a therapeutically effective amount of a composition disclosed herein. Disclosed are methods of decreasing lipids in the liver of a subject comprising administering to the subject a therapeutically effective amount of a composition disclosed herein.

### BACKGROUND

Adipose tissue is the largest endocrine organ in the body accounting on average for 30% of the body weight. Adipose regulates the whole body metabolic processes: energy balance and thermogenesis, glucose and lipid homeostasis, inflammation and immunity. Adipose serves as a stem cell reservoir that is essential for tissue and organ regeneration. Disrupted metabolism in the adipose tissue due to obesity and aging results in inflammation, increase in blood glucose and lipids, insulin resistance and oxidative stress-induced cell death. Adipose dysfunction plays a key role in metabolic syndrome, diabetes and liver diseases.

NAFLD and its progressive form NASH is the most common chronic liver disease and is a leading cause of liver-related morbidity and mortality worldwide. The incidence and prevalence of NAFLD is increasing parallel with obesity growth. NASH significantly increases risks of cirrhosis and hepatocellular carcinoma, leading to liver failure and liver transplant. Currently, there are no NASH specific therapies. Given NASH's high prevalence, the economic and health burden of NASH and NASH-associated morbidity and mortality, there is an unmet medical need for therapies that can stop or slow the progression or reverse NASH and NAFLD.

Although adipose has a therapeutic potential, unfortunately, transplantation of adipose tissue is not necessarily feasible for the treatment of patients due to requirements for a large amount of fat and immunosuppression. The systemic delivery of a mesenchymal stem cell (MSC) suspension does not target the abnormal adipose, the root cause of metabolic abnormalities and an advanced stage of non-alcoholic fatty liver disease (NAFLD) known as NASH. It has also been shown that implanted Poly(lactide-co-glycolide) scaffolds (PLGS) led to decrease in lipid accumulation in muscle and liver via giant cell formation in the scaffold that were metabolizing lipids and had no effect on adipose tissue metabolism. Therefore, there is a need for adipose products for treating metabolic diseases.

Thus, a new and improved approach to adipose products for treating metabolic diseases, such as NASH, is needed.

### BRIEF SUMMARY

Disclosed are compositions comprising an adipose matrix and methods for treatment of NASH and other metabolic diseases with the compositions either alone or in combination with MSCs.

Disclosed are adipose matrices comprising devitalized and partially delipidized adipose tissue. Disclosed are adipose matrices comprising vitalized and partially delipidized adipose tissue.

Disclosed are compositions comprising an adipose matrix comprising devitalized and partially delipidized adipose tissue. Disclosed are compositions comprising an adipose matrix comprising vitalized and partially delipidized adipose tissue.

Disclosed are compositions comprising an adipose matrix comprising devitalized, or vitalized, and partially delipidized adipose tissue and MSCs.

Disclosed are methods of making any of the disclosed adipose matrices or compositions.

Disclosed are methods of treating a subject having a metabolic liver disease, metabolic disease, ischemic wound, or inflammatory disease comprising administering to the subject a therapeutically effective amount of one or more of the compositions disclosed herein.

Disclosed are methods of decreasing cholesterol in a subject comprising administering to the subject a therapeutically effective amount of one or more of the compositions disclosed herein.

Disclosed are methods of decreasing lipids in the liver of a subject comprising administering to the subject a therapeutically effective amount of one or more of the compositions disclosed herein.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows fluorescent microscopic images of Calcein-stained undifferentiated and adipo-differentiated human mesenchymal stem cells (hMSCs) and rat mesenchymal stem cells (rMSCs) one week after cells were seeded on the devitalized partially delipidized human or rat adipose matrix, respectively.

FIG. 2 shows secretion of adiponectin by human MSC (hMSC) and rat MSC (rMSC) formulations (“Matrix+Adipo-MSCs) over time in culture medium in vitro. Adipose matrix alone (“Matrix”) or adipose matrix seeded with non-differentiated MSCs (“Matrix+MSCs) serve as controls.

FIG. 3 shows an evaluation of rat MSC formulation xenograft functionality in nude mice study design.

FIG. 4 shows a visual appearance and weights of rMSC formulations excised from nude mice 3 weeks post-injection

FIG. 5 shows images of Hematoxylin-Eosin (H&E)-stained sections of the rMSC formulation prior and 3 weeks post-injection.

FIG. 6 shows representative images of Hematoxylin-Eosin (H&E)-stained sections of the rMSC formulation structure prior to injection (left image), and 3 weeks post-injection (middle and right images). Images of the rMSC formulation 3 weeks post-injection show new adipocyte formation (middle image) and new blood vessel formation and mouse cell infiltration (right image, red arrows) in the rMSC formulation.

FIG. 7 shows fluorescent images of AO/PI (left images) and AO (right images) stained rMSC formulation excised from nude mice 3 weeks post-injection.

FIG. 8 shows adipogenic differentiation of culture expanded cells that were isolated from rMSC formulation excised from nude mice 3 weeks post-injection.

FIG. 9 shows a development and treatment of Non-Alcoholic SteatoHepatitis (NASH) by the rMSC formulation in obese fa/fa Zucker rats study design.

FIG. 10 shows mean+/−SD body weights from Day 0 to Day 126 for all animals during the study (left graph) and for control versus treated animals during the study (right graph).

FIG. 11 shows liver appearance at the baseline, week 4, week 8, week 15 and week 18 study time points.

FIG. 12 shows liver weight at the end of the study for control and treated animals. Bars are: mean+/−SD.

FIG. 13 shows serum cholesterol at the end of the study for control and treated animals. Bars are: mean+/−SD.

FIG. 14 shows serum adiponectin at the end of the study for control and treated animals. Bars are: mean+/−SD; the dotted horizontal line shows the value of serum adiponectin at the baseline.

FIG. 15 shows Hematoxylin & Eosin (H&E) and Masson's Trichrome (MT) histological staining of liver sections at the baseline, week 4, week 8 and week 15 of the study time points.

FIG. 16 shows representative histological images of Hematoxylin & Eosin (H&E) and Masson's Trichrome (MT) stained liver sections for animals from the control and treatment groups at the end of study (week 18).

FIG. 17 shows liver pathology histological scores for animals from the control and treatment groups at the end of study (week 18). Bars are: mean+/−SD.

FIG. 18 shows visual appearance of the rMSC formulation at the abdominal adipose injection sites for the treated animals at the end of the study (week 18, 3 weeks post-injection).

FIG. 19 shows histological images of Hematoxylin & Eosin (H&E) and Masson's Trichrome (MT) stained rMSC formulation sections for treated animals at the end of study (week 18, 3 weeks post-injection).

FIG. 20 shows representative images of Hematoxylin-Eosin (H&E)-stained sections of the rMSC formulation structure prior to injection (left image), and 3 weeks post-injection (middle and right images). Images of the rMSC formulation 3 weeks post-injection show new adipocyte formation (middle image, in the red circle) and new blood vessel formation and host cell infiltration (right image, red arrows) in the rMSC formulation.

FIG. 21 shows fluorescent images of AO/PI (left images) and AO (right images) stained rMSC formulation excised from Zucker rats 3 weeks post-injection.

FIG. 22 shows the competitive advantage of the compositions described herein vs known drugs for treating NASH.

FIG. 23 shows liver appearance and weight for each animal from the baseline, week 4, week 8 control and treatment groups.

FIG. 24 shows representative images of H&E-stained liver tissue sections in rats B30-106-021 (Baseline group), B30-106-015 (Control, 8 weeks group), B30-106-001 (M1 treated group), B30-106-004 (M1+ cells treated group), B30-106-007 (M2 treated group) and B30-106-011 (M2+ cells treated group). B30-106-021 has normal liver tissue structure. All other sections show steatosis. B30-106-004 and B30-106-011 treated by formulations containing cells developed microsteatosis. B30-106-001 and B30-106-007 animals treated by formulations without cells have both micro- and macrosteatosis. Red stars in liver tissue section from B30-106-015 8 weeks control group animal show examples of macrosteatosis.

FIG. 25 shows representative images of Masson's trichome stained liver tissue sections in rats B30-106-019 (Baseline group), B30-106-015 (8 weeks control group) and B30-106-012 (M2+ cells treated group). Liver sections from B30-106-019 (baseline group) and B30-106-012 (M2+ cells treated group) show normal collagen distribution in the perivenular area. Animal B30-106-015 shows the presence of perivenular and zone 3 fibrosis (score “1”).

FIG. 26 shows histological appearance of the four therapeutic formulations 4 weeks after injection in rats, formulations prior to injection and normal rat white adipose.

FIG. 27 shows representative images of H&E-stained section of each therapeutic formulations 4 weeks after injection in rats (4 left images). Two right images show infiltration with inflammatory cells and giant cell formation observed predominantly in the M1 formulation post-injection (top image) and neo-angiogenesis observed predominantly in the M2 and M2+ cells formulations post-injection (bottom image).

FIG. 28 shows representative images of negative control (left images), CD206 (middle images) and CPT1A (right images) ICH-stained sections of rat adipose (the top row) and formulation #4 prior to injection (the bottom row). Rat adipose show the presence of CD206 positive cells (M2 macrophages) and CPT1A positive cells, which are adipocytes and M2 macrophages. Black arrows point to the positively stained cells in the adipose. Formulation #4 has neither CD206 nor CPT1A positive cells.

FIG. 29 shows representative images of CD206 and CPT1A ICH-stained sections of 4 adipose formulations that were excised from rats 4 weeks post-injection. All 4 formulations show the presence of CD206 and CPT1A positive cells (black arrows).

FIG. 30 shows representative images of CD206 and CPT1A ICH-stained sections of 4 adipose formulations that were excised from rats 4 weeks post-injection. All 4 formulations show the presence of CD206 and CPT1A positive cells (black arrows).

FIG. 31 shows representative images of CD206 and CPT1A ICH-stained sections of adipose formulation #4 that was excised from rats 4 weeks post-injection. The images show several clusters of giant cells that are CD206 negative, but CPT1A highly positive (black arrows).

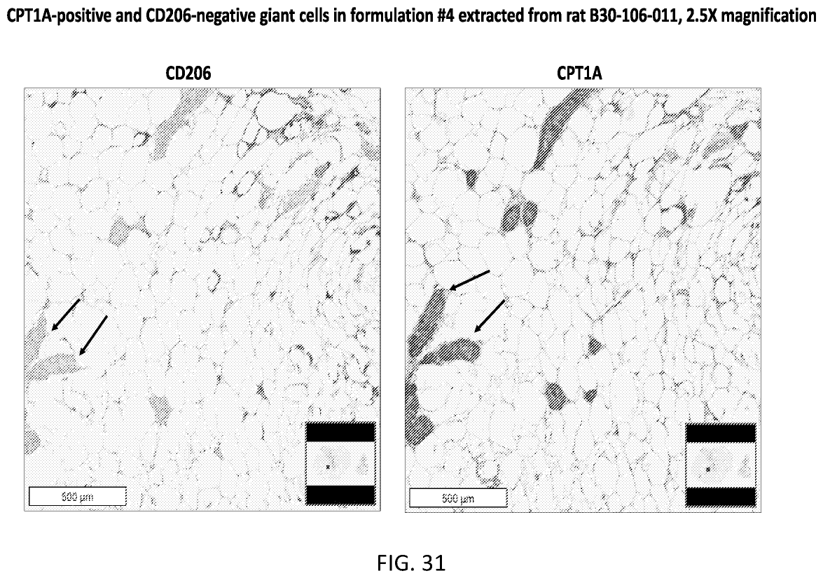

FIGS. 32A-32C show results of in vitro LPS challenge immunogenicity assay development for living donor #1 and for cadaveric donor #3: table with sample description and numerical values (A), results for living donor (B) and results for cadaveric donors (C).

FIG. 33 shows “Evaluation of an inflammatory response against cryopreserved devitalized and viable human adipose tissue in an immune competent mouse model” study design. Ten 8 weeks old female FVB.129P2-Pde60/AntJ mice receive subcutaneous injections of ˜0.25-0.3 mL/point of devitalized and viable cryopreserved human adipose tissue grafts. Gross examination & sample collection (n=3, for 4 weeks—n=4): ½ each graft—fixed for histology; ½ each graft—frozen for cytokine & human cell analysis; Mouse adipose—for histology (reference control)

FIG. 34 shows visual appearance of dissected cryopreserved devitalized and viable human adipose tissue grafts from 4 animals (#1-4) 4 weeks post-implantation.

FIG. 35 shows H&E histological staining of dissected cryopreserved devitalized and viable human adipose tissue grafts from 4 animals (#1-4) 4 weeks post-implantation.

FIG. 36 shows Masson's Trichrome (MT) and Hematoxylin & Eosin (H&E) histological staining of dissected cryopreserved devitalized and viable human adipose tissue grafts 4 weeks post-implantation. M—newly formed normal mouse adipose; H—necrotic human adipose graft

FIG. 37 shows immunohistochemical staining of dissected cryopreserved devitalized and viable human adipose tissue grafts 4 weeks post-implantation at 20× magnification. Mouse 4F/80 marker detects mouse macrophages, mouse CD3 marker detects mouse T-lymphocytes and human KU-80 marker detects the presence of human cells in the adipose grafts.

FIG. 38 shows a schematic diagram of a rat wound model animal study design.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and CE are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a adipose tissue” includes a plurality of such adipose tissues, reference to “the adipose tissue” is a reference to one or more adipose tissues and equivalents thereof known to those skilled in the art, and so forth.

As used herein, the term “subject” or “patient” can be used interchangeably and refer to any organism to which a composition of this invention may be administered, e.g., for experimental, diagnostic, and/or therapeutic purposes. Typical subjects include animals (e.g., mammals such as non-human primates, and humans; avians; domestic household or farm animals such as cats, dogs, sheep, goats, cattle, horses and pigs; laboratory animals such as mice, rats and guinea pigs; rabbits; fish; reptiles; zoo and wild animals). Typically, “subjects” are animals, including mammals such as humans and primates; and the like.

By “treat” is meant to administer an adipose matrix to a subject, such as a human or other mammal (for example, an animal model) that has an increased susceptibility for developing metabolic liver disease, in order to prevent or delay a worsening of the effects of the disease or condition, or to partially or fully reverse the effects of the disease or condition (e.g., cancer). For example, “treat” can mean to prevent disease progression.

By “prevent” is meant to minimize the chance that a subject who has an increased susceptibility for developing a disease or disorder will develop the disease or disorder such as metabolic liver disease.

As used herein, the terms “administering” and “administration” refer to any method of providing an adipose matrix or composition as described herein, to a subject. Such methods are well known to those skilled in the art and include, but are not limited to: subcutaneous administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and oral administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition. In an aspect, the skilled person can determine an efficacious dose, an efficacious schedule, or an efficacious route of administration so as to treat a subject.

By an “effective amount” of a composition as provided herein is meant a sufficient amount of the composition to provide the desired effect. The exact amount required will vary from subject to subject, depending on the species, age, and general condition of the subject, the severity of disease (or underlying genetic defect) that is being treated, the particular composition used, its mode of administration, and the like. Thus, it is not possible to specify an exact “effective amount.” However, an appropriate “effective amount” may be determined by one of ordinary skill in the art using only routine experimentation. The term “therapeutically effective amount” means an amount of a therapeutic, prophylactic, and/or diagnostic agent (e.g., adipose matrix) that is sufficient, when administered to a subject suffering from or susceptible to a specific disease or condition (e.g., metabolic liver disease, metabolic diseases, inflammatory diseases) to treat, alleviate, ameliorate, relieve, alleviate symptoms of, prevent, delay onset of, inhibit progression of, reduce severity of, and/or reduce incidence of the disease or condition.

“Natural”, in the context of, for example, “natural adipose tissue,” refers to properties exhibited by the adipose tissue in its native state in the subject or donor.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinence of the cited documents. It will be clearly understood that, although a number of publications are referred to herein, such reference does not constitute an admission that any of these documents form part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Compositions

As described herein, composition can be used to refer to the adipose matrix alone or the adipose matrix in combination with something else, such as MSCs or a pharmaceutically acceptable carrier.

Disclosed are adipose matrices comprising devitalized and partially delipidized adipose tissue. In some aspects, the adipose tissue can be white adipose or brown adipose.

In some aspects, the term “devitalized” means at some point 100% of the native cells (e.g., cells that originated from the adipose tissue) have been killed and at least a portion of the killed cells (i.e., nonviable cells) are present in the adipose tissue. In some aspects, a devitalized adipose tissue can later be populated with cells; however, if the adipose tissue was initially devitalized then even after populating with cells at a later step the adipose tissue can still be referred to as devitalized. In some aspects, devitalized adipose tissue can mean that some of the dead cells can be lost during processing, but at least 98% of the dead cells are still present in the tissue.

Also disclosed are adipose matrices comprising vitalized and partially delipidized adipose tissue. In some aspects, the adipose tissue can be white adipose or brown adipose. Thus, in some aspects, the description of the devitalized and partially delipidized adipose tissue is the same for the vitalized and partially delipidized adipose tissue, the only difference being one is devitalized and one is vitalized. In some aspects, the rest of the composition can be identical.

In some aspects, the partially delipidized adipose tissue comprises at least 50% of the original lipids. In some aspects, the partially delipidized adipose tissue comprises at least 10%, 15%, 20%, 25%, 30, 35, 40%, 45%, 50%, 55%, 60%, 65%, 70%, 75%, 80%, 85%, or 90% of the original lipids. In some aspects, the partially delipidized adipose tissue comprises less than 100% of the original lipids but more than 5% of the original lipids. Thus, in some aspects, a partially delipidized adipose tissue does not have 100% of the original lipids of the adipose tissue but also is not completely devoid of lipids.

In some aspects, the devitalized and partially delipidized adipose tissue can be minced or micronized. In some aspects, the vitalized and partially delipidized adipose tissue can be minced or micronized. In some aspects, the minced adipose tissue comprises pieces of adipose tissue less than 1 mm in size. In some aspects, the minced or micronized adipose tissue is a homogenous population of pieces of adipose tissue less than 1 mm in size. In some aspects, the minced or micronized adipose tissue is a non-homogenous population of pieces of adipose tissue less than 1 mm in size.

In some aspects, the adipose tissue comprises all of the components of native adipose tissue except for a portion of the lipids and some cells. In some aspects, the native adipose tissue factors that can provide therapeutic effects include, but are not limited to, adiponectin, leptin, vascular endothelial growth factors (VEGFs), platelet-derived growth (PDGFs), fibroblast growth Factors (FGFs), IL-6, IL-8, insulin like growth factors (IGFs), and hepatocyte growth factor (HGF).

Disclosed are compositions comprising any of the disclosed adipose matrices. For example, disclosed are compositions comprising an adipose matrix comprising devitalized and partially delipidized adipose tissue or compositions comprising an adipose matrix comprising vitalized and partially delipidized adipose tissue.

In some aspects, the composition further comprises mesenchymal stem cells (MSCs). For example, disclosed are compositions comprising an adipose matrix comprising devitalized and partially delipidized adipose tissue and MSCs or compositions comprising an adipose matrix comprising vitalized and partially delipidized adipose tissue and MSCs. In some aspects, the MSCs can be differentiated, undifferentiated, or a combination thereof. In some aspects, the MSCs are adipogenic differentiated MSCs. In some aspects, the MSCs are a combination of undifferentiated MSCs and adipogenic differentiated MSCs. In some aspects, the MSCs have been cultured prior to combining with the adipose matrix. In some aspects, the MSC formulations can be referred to as M2+ cells or BRC001.

In some aspects, the MSCs are allogeneic to the adipose tissue. For example, the adipose tissue can be derived from a first subject and the MSCs derived from a second subject, wherein the first and second subjects are not the same subject. In some aspects, the MSCs are autologous to the adipose tissue. For example, the adipose tissue and the MSCs can be derived from the same subject. In some aspects, MSCs can be isolated (and in some instances expanded) from an adipose tissue and then after devitalizing and delipidizing the adipose tissue the MSCs can be added back to the tissue. Thus, the MSCs can be autologous. In some aspects, the MSCs can be from two or more subjects. In some aspects, the MSCs can be a combination of both autologous and allogeneic MSCs.

In some aspects, the cells can be originally derived from the adipose tissue but still exogenously added to the adipose matrix. For example, MSCs can be removed from the adipose tissue, then the adipose tissue can be devitalized and/or partially delipidized and then the MSCs can be added back to the adipose matrix. In this scenario, the MSCs are native to the adipose matrix as they are the tissues own naturally derived cells, but they are still exogenously added. In some aspects, the adipose matrix has no native cells present. Adipose matrix that has no native cells present means there are no cells from the original adipose tissue that are still present. Thus, any viable cells present in the adipose matrix would be from a different tissue or subject.

In some aspects, the adipose tissue is mammalian. In some aspects, the MSCs are mammalian.

In some aspects, the MSCs can be from adipose or any other source. For example, in some aspects, the MSCs can be from, but are not limited to, placenta, bone marrow or bone.

In some aspects, the MSCs are present in an amount of 5×10⁵ to 50×10⁶ MSCs per 1 milliliter of devitalized, or vitalized, and partially delipidized adipose tissue. In some aspects, the adipose matrix comprises about 0.25-20 μg DNA per mg of adipose matrix.

### 1. Pharmaceutical Compositions

Disclosed are pharmaceutical compositions comprising any of the adipose matrices or compositions disclosed herein and a pharmaceutically acceptable carrier. For example, disclosed are pharmaceutical compositions comprising a composition comprising an adipose matrix comprising devitalized, or vitalized, and partially delipidized adipose tissue and a pharmaceutically acceptable carrier.

Disclosed herein are pharmaceutical compositions that comprise one or more of the compositions disclosed herein. In an aspect, the pharmaceutical composition can comprise an adipose matrix comprising devitalized, or vitalized, and partially delipidized adipose tissue. In an aspect, the pharmaceutical composition can comprise an adipose matrix comprising devitalized, or vitalized, and partially delipidized adipose tissue and MSCs.

In some aspects, the pharmaceutical compositions can further comprise a pharmaceutically acceptable carrier. In some aspects, the pharmaceutical compositions described herein can be sterile and contain any of the disclosed compositions for producing the desired response in a unit of weight or volume suitable for administration to a subject. In some aspects, the pharmaceutical compositions can contain suitable buffering agents, including, e.g., acetic acid in a salt; citric acid in a salt; boric acid in a salt; and phosphoric acid in a salt.

When administered, the disclosed compositions or pharmaceutical compositions can be administered in pharmaceutically acceptable preparations. Such preparations may routinely contain pharmaceutically acceptable concentrations of salt, buffering agents, preservatives, compatible carriers, supplementary immune potentiating agents such as adjuvants and cytokines, and optionally other therapeutic agents.

As used herein, the term “pharmaceutically acceptable” means a non-toxic material that does not interfere with the effectiveness of the biological activity of the active ingredients. The term “physiologically acceptable” refers to a non-toxic material that is compatible with a biological system such as a cell, cell culture, tissue, or organism. The characteristics of the carrier will depend on the route of administration. Physiologically and pharmaceutically acceptable carriers include diluents, fillers, salts, buffers, stabilizers, solubilizers, and other materials which are well known in the art. The term denotes an organic or inorganic ingredient, natural or synthetic, with which the active ingredient is combined to facilitate the application. The components of the pharmaceutical compositions also are capable of being co-mingled with the disclosed compositions, and with each other, in a manner such that there is no interaction which would substantially impair the desired pharmaceutical efficacy.

As used herein, the term “pharmaceutically acceptable carrier” refers to solvents, dispersion media, coatings, antibacterial, isotonic and absorption delaying agents, buffers, excipients, binders, lubricants, gels, surfactants that can be used as media for a pharmaceutically acceptable substance. The pharmaceutically acceptable carriers can be lipid-based or a polymer-based colloid. Examples of colloids include liposomes, hydrogels, microparticles, nanoparticles and micelles. The compositions can be formulated for administration by any of a variety of routes of administration, and can include one or more physiologically acceptable excipients, which can vary depending on the route of administration. Any of the compositions described herein can be administered in the form of a pharmaceutical composition.

As used herein, the term “excipient” means any compound or substance, including those that can also be referred to as “carriers” or “diluents.” Preparing pharmaceutical and physiologically acceptable compositions is considered routine in the art, and thus, one of ordinary skill in the art can consult numerous authorities for guidance if needed. The compositions can also include additional agents (e.g., preservatives).

The pharmaceutical compositions disclosed herein can be sterile and sterilized by conventional sterilization techniques or sterile filtered. Aqueous solutions can be packaged for use as is, or lyophilized, the lyophilized preparation, which is encompassed by the present disclosure, can be combined with a sterile aqueous carrier prior to administration. The pH of the pharmaceutical compositions typically will be between 3 and 11 (e.g., between about 5 and 9) or between 6 and 8 (e.g., between about 7 and 8). The resulting compositions in solid form can be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment. The compositions can also be formulated as powders, elixirs, suspensions, emulsions, solutions, syrups, aerosols, lotions, creams, ointments, gels, suppositories, sterile injectable solutions and sterile packaged powders. The active ingredient can be any of the growth hormone releasing hormone peptides described herein in combination with one or more pharmaceutically acceptable carriers. As used herein “pharmaceutically acceptable” means molecules and compositions that do not produce or lead to an untoward reaction (i.e., adverse, negative or allergic reaction) when administered to a subject as intended (i.e., as appropriate).

In some aspects, administration of disclosed compositions or pharmaceutical compositions disclosed herein can be administered to mammals other than humans, e.g., for testing purposes or veterinary therapeutic purposes, can be carried out under substantially the same conditions as described above.

### 2. Cryopreserved or Lyophilized Compositions

In some aspects, the disclosed adipose matrices can be cryopreserved. In some aspects, the disclosed adipose matrices can further comprise a cryopreservation solution. In some aspects, the disclosed adipose matrices can be previously cryopreserved. In some aspects, the disclosed compositions or pharmaceutical compositions can be cryopreserved. In some aspects, the disclosed compositions or pharmaceutical compositions can further comprise a cryopreservation solution. In some aspects, the disclosed compositions or pharmaceutical compositions can be previously cryopreserved. In some aspects, the disclosed MSCs can be cryopreserved. In some aspects, the disclosed MSCs can further comprise a cryopreservation solution. In some aspects, the disclosed MSCs can be previously cryopreserved. “Previously cryopreserved” can mean a composition that was once cryopreserved but has been removed (e.g., thawed) from the cryopreserved state.

Thus, for example, disclosed are compositions comprising an adipose matrix comprising devitalized, or vitalized, and partially delipidized adipose tissue and further comprising a cryopreservation solution. A further example includes, compositions comprising an adipose matrix comprising devitalized, or vitalized, and partially delipidized adipose tissue and MSCs, and further comprising a cryopreservation solution.

Disclosed are cryopreserved adipose matrices and compositions prepared using the disclosed methods of micronizing adipose tissue; emulsifying the micronized adipose tissue; centrifuging the micronized adipose tissue to produce at least two distinct layers, a top layer and a layer directly underneath the top layer, wherein free lipids are present in the top layer and a partially delipidized adipose tissue is present in the layer directly underneath the top layer; removing the free lipids and freezing the adipose tissue before or after any of these steps to devitalize the adipose tissue. In some aspects, a third layer is present beneath the layer comprising the partially delipidized adipose tissue. In some aspects, the third layer comprises PBS from the wash.

Disclosed herein are adipose matrices comprising devitalized, or vitalized, and partially delipidized adipose tissue. Disclosed herein are cryopreserved compositions comprising an adipose matrix comprising devitalized and partially delipidized adipose tissue. Disclosed herein are cryopreserved compositions comprising an adipose matrix comprising devitalized and partially delipidized adipose tissue and MSCs. In some aspects, both the adipose matrix and the MSCs are cryopreserved in a single composition. In some aspects, the adipose matrix and the MSCs are cryopreserved in multiple compositions and later combined into a single composition.

In some aspects, the cryopreserved adipose matrices and/or compositions comprise at least 10% native lipids. In some aspects, the cryopreserved adipose matrix or composition, when thawed, can comprise at least 10% lipids. In some aspects, the cryopreserved or previously cryopreserved adipose matrices or composition can comprise greater than 10%, 15%, 20%, 25%, 30%, 35%, 40%, 45%, 50%, 55%, 60%, 65%, 70%, 75%, 80%, 85%, 90%, 95%, or 99% lipids. In some aspects, the cryopreserved or previously cryopreserved adipose matrix or composition can be cut to a desired size. The percent of lipids present after thawing is at least 10, 20, 30, 40, 50, 60, 70, 80, 90, or 100% of the lipids present immediately prior to cryopreservation (but after the partial delipidization step).

In some aspects, a cryopreservation solution can contain one or more non-cell permeating cryopreservatives. Examples of non-cell permeating cryopreservatives, include but not limited to, polyvinyl pyrrolidione, a hydroxyethyl starch, a polysaccharide, a monosaccharide, an alginate, trehalose, raffinose, dextran, human serum albumin, Ficoll, lipoproteins, polyvinyl pyrrolidone, hydroxyethyl starch, autologous plasma or a mixture thereof. In some aspects, the cryopreservative does not contain DMSO or glycerol. Further, a cryopreservation solution can contain serum albumin or other suitable proteins to stabilize the disclosed compositions during the freeze-thaw process and to reduce the damage to cells, thereby maintaining viability. In some aspects, a cryopreservation solution can contain a physiological solution, such as a physiological buffer or saline, for example phosphate buffer saline. In some aspects, a cryopreservation solution can comprise a lyoprotectant, such as trehalose or trehalose in combination with one or more antioxidants.

In some aspects, the disclosed compositions can be lyophilized.

Disclosed are lyophilized adipose matrices comprising devitalized and partially delipidized adipose tissue. Disclosed are lyophilized compositions comprising an adipose matrix comprising devitalized and partially delipidized adipose tissue. Also disclosed are lyophilized compositions comprising an adipose matrix comprising devitalized and partially delipidized adipose tissue and MSCs.

Disclosed are lyophilized adipose matrices and compositions prepared using the disclosed methods of micronizing adipose tissue; emulsifying the micronized adipose tissue; centrifuging the micronized adipose tissue to produce at least two distinct layers, a top layer and a layer directly underneath the top layer, wherein free lipids are present in the top layer and a partially delipidized adipose tissue is present in layer directly underneath the top layer; removing the free lipids, freezing the adipose tissue before or after any of these steps to devitalize the adipose tissue, and lyophilizing the partially delipidized and devitalized adipose matrix. In some aspects, a third layer is present beneath the layer comprising the partially delipidized adipose tissue. In some aspects, the third layer comprises PBS from the wash.

In some aspects, the disclosed lyophilized adipose matrices or compositions comprise less than 15% residual water. In some aspects, the disclosed lyophilized adipose matrices or compositions comprise 5-12% residual water. In some aspects, the disclosed lyophilized adipose matrices or compositions comprise ≤5% residual water.

In some aspects, the disclosed lyophilized adipose matrices or compositions comprise trehalose. In some aspects, the disclosed lyophilized adipose matrices or compositions comprise trehalose, wherein the trehalose is present at a concentration of 0.25M-1.5M.

In some aspects, the lyophilized composition, when reconstituted can comprise at least 70% viable MSCs compared to the amount of MSCs prior to lyophilizing. In some aspects, reconstituted tissue can comprise greater than 40%, 45%, 50%, 55%, 60%, 65%, 70%, 75%, 80%, 85%, 90%, 95%, or 99% of the MSCs present prior to lyophilization. In some aspects, after reconstituting the lyophilized tissue, the tissue can then be cut to a desired size.

In some aspects, the lyophilized adipose matrices or compositions disclosed herein can be stable for at least three weeks. In some aspects, the lyophilized adipose matrices or compositions can be stable for at least three months. In some aspects, the lyophilized compositions can be stable for 1, 2, 3, 4, 5, 6, 7, 8, 9, 10, 11, 12, 24, 36, 48, or 60 months.

In some aspects, the lyophilized compositions disclosed herein can be reconstituted resulting in a reconstituted tissue. The described lyophilized adipose matrices or compositions can be reconstituted using standard techniques known in the art. In some aspects, reconstituting refers to rehydrating. Thus, the disclosed lyophilized adipose matrices or compositions can be reconstituted or rehydrated using water, saline, a buffer such as, but not limited to phosphate buffered saline (PBS), in a solution comprising a stabilizing agent such as, but not limited to bovine serum albumin (BSA), Plasma-Lyte A or other clinically available electrolyte solutions, with human bodily fluids or a combination thereof. For example, lyophilized adipose matrices or compositions can be applied directly to a wound or tissue injury on a subject and the subject's bodily fluids can reconstitute. In some aspects, a combination of bodily fluids and another known rehydrating solution can be used. Also, disclosed are reconstituted adipose matrices or compositions prepared using the methods disclosed herein.

### C. Methods of Making

Disclosed are methods of making any of the disclosed adipose matrices, compositions or pharmaceutical compositions.

Disclosed are methods comprising micronizing adipose tissue; emulsifying the adipose tissue; centrifuging, after emulsifying, the adipose tissue to produce at least two distinct layers, a top layer and a layer directly underneath the top layer, wherein free lipids are present in the top layer and partially delipidized adipose tissue is present in the layer directly underneath the top layer; removing the top layer, freezing the adipose tissue before or after micronizing or emulsifying, wherein the combination of these steps results in an adipose matrix comprising devitalized and partially delipidized adipose tissue. In some aspects, the method results in a composition comprising an adipose matrix comprising devitalized and partially delipidized adipose tissue. XXX THESE ARE THE STEPS FOR MAKING DEVITALIZED. PLEASE PROVIDE A SMALL WRITE UP SIMILAR TO THIS FOR MAKING VITALIZED AND PARTIALLY DELIPIDIZED XXX

Disclosed are methods comprising micronizing adipose tissue; partially emulsifying the adipose tissue; centrifuging, after emulsifying, the adipose tissue to produce at least two distinct layers, a top layer and a layer directly underneath the top layer, wherein free lipids are present in the top layer and partially delipidized adipose tissue is present in the layer directly underneath the top layer; removing the top layer, cryopreserving the adipose tissue after micronizing and emulsifying, wherein the combination of these steps results in an adipose matrix comprising vitalized and partially delipidized adipose tissue. In some aspects, the method results in a composition comprising an adipose matrix comprising vitalized and partially delipidized adipose tissue.

In some aspects, the difference in the method of making devitalized, partially delipidized adipose and making vitalized, partially delipidized can be in the number of passages and the amount of applied force during emulsifying. In some aspects, the difference in the method of making devitalized, partially delipidized adipose and making vitalized, partially delipidized can be devitalized samples are frozen while the vitalized samples are cryopreserved.

In some aspects, the method comprises obtaining adipose tissue prior to micronizing or emulsifying the adipose tissue. In some aspects, the obtained adipose tissue is frozen or has been previously frozen. If the obtained adipose tissue is frozen or has been previously frozen, the disclosed methods can be performed without the freezing step. In some aspects, the step of freezing the adipose tissue allows for devitalization of the adipose tissue. Thus, upon freezing, the cells present in the adipose tissue, such as stromal cells and immune cells, will be killed. The dead cells can remain in the adipose tissue, thus the adipose tissue can be referred to as devitalized, not decellularized or not acellular. In some aspects, the adipose tissue can be decellularized. In some aspects, the adipose tissue can maintain 5, 10, 20, 30, 40, 50, 60, 70, 80, 90, or 99% of its viable cells, thus being vitalized.

In some aspects, the step of micronizing allows for the adipose tissue to be turned into many small pieces. In some aspects, the micronizing of the adipose tissue produces a population of adipose tissue pieces less than 1 mm in size each. In some aspects, micronizing can result in a homogenous population of pieces. In some aspects, the micronizing results in a population of adipose tissue pieces of varying sizes. In some aspects, this can help with making the adipose matrix injectable through a syringe into a subject. In some aspects, micronizing and mincing can be used interchangeably. In some aspects, the micronizing can be performed manually or mechanistically. If freezing of the adipose tissue has occurred prior to micronizing, then after micronizing the result is a micronized, devitalized adipose tissue. In some aspects, micronizing can occur after emulsifying. In some aspects, micronizing can occur after freezing.

In some aspects, the step of emulsifying helps kill adipocytes. Thus, in some aspects, the emulsifying partially devitalizes the adipose tissue. In some aspects, the destruction of adipocytes can help release lipids from the adipose tissue. The step of centrifuging results in a top layer (of free lipids) and a layer directly underneath the top layer (of partially delipidized adipose tissue). Therefore, centrifuging the adipose tissue after emulsifying causes the free lipids to be present in the top layer and the partially delipidized adipose tissue to be present in the layer directly underneath the top layer. In some aspects, the partially delipidized adipose tissue comprises at least 50% of the original lipids. In some aspects, the partially delipidized adipose tissue comprises at least 10%, 15%, 20%, 25%, 30, 35, 40%, 45%, 50%, 55%, 60%, 65%, 70%, 75%, 80%, 85%, or 90% of the original lipids. In some aspects, the partially delipidized adipose tissue comprises less than 100% of the original lipids but more than 5% of the original lipids. Thus, in some aspects, a partially delipidized adipose tissue does not have 100% of the original lipids of the adipose tissue but also is not completely devoid of lipids. If freezing of the adipose tissue has occurred any time before the emulsifying step then the pellet comprises a partially delipidized and a devitalized adipose tissue. In some aspects, the freezing can occur to the layer directly underneath the top layer. In some aspects, a third layer is present beneath the layer comprising the partially delipidized adipose tissue. In some aspects, the third layer comprises PBS from the wash.

In some aspects, emulsifying can be performed using any known technique. For example, a homogenizer can be used or repeatedly passing the adipose tissue through one or more syringes.

In some aspects, multiple freezing steps can occur to ensure devitalization. Thus, the freezing step can occur at one or more of the steps in the methods described herein.

In some aspects, the methods of making any of the disclosed adipose matrices, compositions or pharmaceutical compositions can further comprise adding MSCs. In some aspects, the MSCs can be added at any step after the freezing step. In some aspects, the freezing step can kill the MSCs. For example, in some aspects, the MSCs are added at the very end to the adipose matrix comprising a partially delipidized and devitalized adipose tissue.

In some aspects, the MSCs can be differentiated, undifferentiated, or a combination thereof. In some aspects, the MSCs are adipogenic differentiated MSCs. In some aspects, the MSCs are a combination of undifferentiated MSCs and adipogenic differentiated MSCs. In some aspects, the MSCs have been cultured prior to combining with the adipose matrix. Thus, in some aspects, the methods can further comprise culturing MSCs (whether autologous or allogeneic) prior to combining with the adipose matrix. In some aspects, the culturing occurs in adipogenic differentiation medium.

In some aspects, the MSCs are autologous to the adipose tissue. For example, the adipose tissue and the MSCs can be derived from the same subject. Thus, in some aspects, the methods comprise removing, or isolating, MSCs from the adipose tissue any time prior to freezing. The method can then further comprise adding the MSCs previously removed from the adipose tissue back to the adipose tissue any time after the freezing step. Thus, the MSCs can be autologous. In some aspects, a portion of the adipose tissue can be used to isolate MSCs and a portion of the adipose tissue can be used for delipidizing and devitalizing as disclosed in the methods of making. Then, the isolated MSCs isolated, and in some instances MSCs expanded in culture, can be added to the adipose tissue at any step of the disclosed methods after freezing of the adipose tissue.

In some aspects, the MSCs are allogeneic to the adipose tissue. For example, the adipose tissue can be derived from a first subject and the MSCs derived from a second subject, wherein the first and second subjects are not the same subject. Thus, in some aspects, the methods comprise obtaining MSCs, wherein the MSCs are from a subject different from the subject of the adipose tissue. The method further comprises adding the MSCs to the adipose tissue any time after the freezing step.

In some aspects, the MSCs can be from a pool of two or more subjects. In some aspects, the MSCs can be a combination of both autologous and allogeneic MSCs.

In some aspects, the methods further comprise a step of cryopreservation. In some aspects, the methods further comprise a step of lyophilization. In some aspects, the step of cryopreservation or lyophilization can occur before or after adding the MSCs to the adipose tissue.

In some aspects, the MSCs are added to the adipose tissue in an amount of 5×10⁵ to 50×10⁶ MSCs per 1 milliliter of devitalized and partially delipidized adipose tissue. In some aspects, the adipose matrix comprises about 0.25-20 μg DNA per mg of adipose matrix.

### D. Methods of Use

Disclosed are methods of treating a subject having a metabolic liver disease with one or more of the disclosed adipose matrices, compositions or pharmaceutical compositions disclosed herein. Disclosed are methods of treating a subject having a metabolic liver disease comprising administering to the subject a therapeutically effective amount of an adipose matrices, composition or pharmaceutical composition disclosed herein. Disclosed are methods of treating a subject having a metabolic liver disease comprising administering to the subject a therapeutically effective amount of an adipose matrix comprising devitalized and partially delipidized adipose tissue. Disclosed are methods of treating a subject having a metabolic liver disease comprising administering to the subject a therapeutically effective amount of a composition comprising an adipose matrix comprising devitalized and partially delipidized adipose tissue. In some aspects, the composition further comprises mesenchymal stem cells (MSCs). In some aspects, the metabolic liver disease can be, but is not limited to, fatty liver disease or Non-Alcoholic SteatoHepatitis (NASH).

Disclosed are methods of treating a subject having a metabolic disease comprising administering to the subject a therapeutically effective amount of an adipose matrices, composition or pharmaceutical composition disclosed herein. Disclosed are methods of treating a subject having a metabolic disease comprising administering to the subject a therapeutically effective amount of an adipose matrix comprising devitalized and partially delipidized adipose tissue. Disclosed are methods of treating a subject having a metabolic disease comprising administering to the subject a therapeutically effective amount of a composition comprising an adipose matrix comprising devitalized and partially delipidized adipose tissue. In some aspects, the composition further comprises mesenchymal stem cells (MSCs). In some aspects, the metabolic disease can be, but is not limited to, dyslipidemia/hyperlipidemia (for example, subjects with high cholesterol), metabolic syndrome, obesity, type 2 diabetes, insulin resistance, or lipodystrophy.

Disclosed are methods of treating a subject having an ischemic injury comprising administering to the subject a therapeutically effective amount of an adipose matrices, composition or pharmaceutical composition disclosed herein. Disclosed are methods of treating a subject having an ischemic injury comprising administering to the subject a therapeutically effective amount of an adipose matrix comprising devitalized and partially delipidized adipose tissue. Disclosed are methods of treating a subject having an ischemic injury comprising administering to the subject a therapeutically effective amount of a composition comprising an adipose matrix comprising devitalized and partially delipidized adipose tissue. In some aspects, the composition further comprises mesenchymal stem cells (MSCs). In some aspects, the disclosed adipose matrices and compositions can be used to return blood flow to the ischemic injury due to its angiogenic properties.

Disclosed are methods of treating a subject having an inflammatory disease comprising administering to the subject a therapeutically effective amount of an adipose matrices, composition or pharmaceutical composition disclosed herein. Disclosed are methods of treating a subject having an inflammatory disease comprising administering to the subject a therapeutically effective amount of an adipose matrix comprising devitalized and partially delipidized adipose tissue. Disclosed are methods of treating a subject having an inflammatory disease comprising administering to the subject a therapeutically effective amount of a composition comprising an adipose matrix comprising devitalized and partially delipidized adipose tissue. In some aspects, the composition further comprises mesenchymal stem cells (MSCs). In some aspects, the inflammatory disease can be, but is not limited to, autoimmune diseases, inflammation caused by infection, sepsis, any Th1 disease with an excess of TNF. In some aspects, Th1 disease can be, but are not limited to, Crohn's, type 1 diabetes, ulcerative colitis, multiple sclerosis, Hashimoto's thyroiditis, rheumatoid arthritis, psoriasis, alopecia areata, uveitis, Sjogren's syndrome, dermatitis, graft versus host disease. Tolerance induction and organ rejection can also be treated administering to the subject a therapeutically effective amount of a composition disclosed herein. In some aspects, the disclosed adipose matrices and compositions can be used to provide an anti-inflammatory response by activating M2 macrophages.

Disclosed are methods of decreasing cholesterol in a subject comprising administering to the subject a therapeutically effective amount of an adipose matrices, composition or pharmaceutical composition disclosed herein. Disclosed are of decreasing cholesterol in a subject comprising administering to the subject a therapeutically effective amount of an adipose matrix comprising devitalized and partially delipidized adipose tissue. Disclosed are methods of decreasing cholesterol in a subject comprising administering to the subject a therapeutically effective amount of a composition comprising an adipose matrix comprising devitalized and partially delipidized adipose tissue. In some aspects, the composition further comprises mesenchymal stem cells (MSCs). In some aspects, decreasing cholesterol using the disclosed compositions occurs by M2 macrophages having high metabolic activity and oxidizing (burning) fatty acids; formation of new adipocytes that oxidize lipids; activation of metabolic activity of a subject's adipocytes causing upregulation of lipid oxidation; and/or formation of brown-like adipose in white adipose (so called “browning” of white fat—“beige adipocytes”).

Disclosed are methods of decreasing lipids in a subject comprising administering to the subject a therapeutically effective amount of adipose matrices, composition or pharmaceutical composition disclosed herein. Disclosed are methods of decreasing lipids in a subject comprising administering to the subject a therapeutically effective amount of an adipose matrix comprising devitalized and partially delipidized adipose tissue. Disclosed are methods of decreasing lipids in f a subject comprising administering to the subject a therapeutically effective amount of a composition comprising an adipose matrix comprising devitalized and partially delipidized adipose tissue. In some aspects, the composition further comprises mesenchymal stem cells (MSCs). In some aspects, decreasing lipids it can occur through the same mechanisms described above for decreasing cholesterol. In some aspects, decreasing lipids in a subject comprises decreasing blood and/or liver lipids. Thus, disclosed are methods of decreasing blood lipids and lipid accumulation in the liver.

Disclosed are methods of decreasing high fat, cholesterol, and fructose diet-induced lipid oxidation in a subject comprising administering to the subject a therapeutically effective amount of adipose matrices, composition or pharmaceutical composition disclosed herein. Disclosed are methods of decreasing high fat, cholesterol, and fructose diet-induced lipid oxidation in a subject comprising administering to the subject a therapeutically effective amount of an adipose matrix comprising devitalized and partially delipidized adipose tissue. Disclosed are methods of decreasing high fat, cholesterol, and fructose diet-induced lipid oxidation in a subject comprising administering to the subject a therapeutically effective amount of a composition comprising an adipose matrix comprising devitalized and partially delipidized adipose tissue. In some aspects, the composition further comprises mesenchymal stem cells (MSCs).

Disclosed are methods of decreasing liver enzymes in blood in a subject comprising administering to the subject a therapeutically effective amount of adipose matrices, composition or pharmaceutical composition disclosed herein. Disclosed are methods of decreasing liver enzymes in blood in a subject comprising administering to the subject a therapeutically effective amount of an adipose matrix comprising devitalized and partially delipidized adipose tissue. Disclosed are methods of decreasing liver enzymes in blood in a subject comprising administering to the subject a therapeutically effective amount of a composition comprising an adipose matrix comprising devitalized and partially delipidized adipose tissue. In some aspects, the composition further comprises mesenchymal stem cells (MSCs). In some aspects liver enzymes in blood can be a marker for liver damage and therefore decreasing liver enzymes can be a good therapeutic.

In some aspects of any of the disclosed methods, the composition can be administered subcutaneously. For example, subcutaneously can include administering in the subcutaneous fat. In some aspects, the composition can be administered using any known technique for administering therapeutics to a subject. In some aspects, the composition can be administered using any of the routes of administration described herein.

In some aspects of any of the disclosed methods, the adipose matrix or composition comprising the adipose matrix can be any of those described herein.

In some aspects, the adipose matrix can be derived from the adipose of the subject who is being administered the composition. In some aspects, the adipose matrix can be derived from the adipose of a subject different from the subject who is being administered the composition. In some aspects, the MSCs present in a composition comprising adipose matrix and MSCs can be from the subject who is being administered the composition. In some aspects, the MSCs present in a composition comprising adipose matrix and MSCs can be from one or more subjects who are different than the one being administered the composition. In some aspects, the MSCs present in a composition comprising adipose matrix and MSCs can be a combination of MSCs from the subject who is being administered the composition and from one or more subjects different from the one being administered the composition.

In some aspects, the adipose matrix can be derived from a cadaver.

In some aspects, the adipose matrix can be infiltrated with macrophages and/or MSCs derived from the subject.

In some aspects, the MSCs, whether the MSCs in the composition administered to the subject or MSCs from the subject that infiltrate the adipose matrix once administered to the subject, can allow for paracrine secretion of growth factors and cytokines and differentiation into adipocytes, thus providing therapeutic effects. M2 macrophages are known in the art to have protective effects from obesity. Thus, in some aspects, the presence of MSCs resulting in newly formed adipocytes and M2 macrophages protecting from things like obesity are therapeutic effects of the disclosed methods.

In some aspects, new blood vessels are formed in the adipose matrix after administration to the subject. In some aspects, angiogenic growth factors present in the disclosed adipose matrices and compositions can attract endothelial cells. Also, M2 macrophages can be pro-angiogenic as they secrete growth factors to stimulate new blood vessel formation. Thus, in some aspects, the M2 macrophages populate the injected adipose matrices or compositions and induce blood vessel formation.

The methods of use described here provide examples of methods of using adipose matrix comprising devitalized and partially delipidized adipose tissue. Each of these methods can also be performed by administering a composition comprising adipose matrix comprising devitalized and partially delipidized adipose tissue.

### 1. Delivery of Compositions

The disclosed adipose matrices and compositions comprising the adipose matrices can be delivered using a variety of well-known techniques.

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for optical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids, or binders may be desirable. Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mon-, di-, trialkyl and aryl amines and substituted ethanolamines.

### E. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example, disclosed are kits comprising one or more of adipose tissue, and adipose matrix as described herein, and MSCs. The kits also can contain instructions for making the disclosed compositions.

### EXAMPLES

Non-alcoholic fatty liver disease (NAFLD) is the most common chronic liver disease. It is often associated with obesity, type 2 diabetes and metabolic syndrome. NASH is an advanced stage of NAFLD characterized by liver inflammation and fibrosis. According to the American Liver Foundation 16.4 mil patients in the US are affected by NASH, and NASH is expected to be the most frequent reason for liver transplants in the United States by 2030. Currently there are no approved drugs for NASH.

Several lines of evidence support our hypothesis that a combination of mesenchymal stem cells with adipose matrix may have a therapeutic effect for NASH and other diseases characterized by metabolic abnormalities: 1. Transplantation of healthy adipose in obese animals restores disrupted metabolism resulting in lowering body weight, blood glucose and lipids, insulin resistance and abnormalities in liver. 2. Mesenchymal stem cells (MSCs) have biological activities desirable for NASH, and injecting MSCs in animals on a high fat diet reverse negative effects of such diet. 3. Surgical implantation of Poly(lactide-co-glycolide) scaffolds (PLGS) into the epididymal fat of mice fed a high fat diet decreased body weight and ectopic lipid accumulation in muscles and liver. However, transplantation of adipose tissue is not feasible for the treatment of patients due to requirements for a large amount of fat and immunosuppression; systemic delivery of MSC suspension does not target the abnormal adipose, the root cause of metabolic abnormalities and NASH; and implanted PLGS led to giant cell formation in the scaffold that were metabolizing lipids and had no effect on adipose tissue metabolism.

The approach discussed herein for treatment of NASH and other metabolic diseases utilizes the therapeutic properties of a combination of MSCs and adipose matrix delivered directly in subcutaneous adipose in the body of animals and patients. Examples below present data supporting the therapeutic properties of a MSC formulation comprised of stem cells and tissue (matrix with signals). The tissue serves as a stem cell carrier and provides a cell-friendly microenvironment to support stem cell functionality. This can be an off-the-shelf therapy that: has prolonged storage, injectable formulation for minimally invasive delivery in office settings, and no matching or immunosuppression required.

### A. Example 1. Fabrication of Human and Rat MSC Formulation

### 1. Methods and Materials

Human adipose tissues were received from eligible adult living and cadaveric donors after obtaining written informed consent, and tissue regulations for receipt and disposition of tissues was strictly followed.

Rat subcutaneous abdominal adipose tissue was obtained as a part of animal studies. These studies were conducted in compliance with the current version of the following: 1) Animal Welfare Act Regulations (9 CFR); 2) U.S. Public Health Service Office of Laboratory Animal Welfare (OLAW) Policy on Humane Care and Use of Laboratory Animals; 3) Guide for the Care and Use of Laboratory Animals (Institute of Laboratory Animal Resources, Commission on Life Sciences, National Research Council, 1996); and 4) AAALACi accreditation. Procedures used in this study have been designed to avoid or minimize unacceptable discomfort, distress or pain to the animals.

i. Devitalized and Partially Delipidized Human and Rat Adipose Matrix Preparation.

Adipose tissue was cut using scissors and a scalpel into small (3-5 mm) pieces and washed with a large volume of PBS. Then, washed adipose was micronized using a “mezzaluna” knife until the tissue became a homogeneous mass with a <1 mm tissue particle size. Emulsification of micronized adipose tissue was performed by passing tissues 40-50 times between two syringes connected via the Luer Lock connector. During the passing between two syringes the share force results in destruction of adipocytes in adipose matrix leading to release of free lipids. The emulsified adipose was centrifuged at room temperature at 3000 rpm for 5 min. Free lipids from the top were removed with a pipet, and partially delipidized adipose was aliquoted in 1-3 mL portions in syringes and stored at −80° C. prior to use.

In some aspects, the method of making nanofat is similar to that of the partially delipidized adipose matrix, however, nanofat is not micronized, just emulsified, and is then filtered through tiny nylon mesh to remove pieces of tissue. Nanofat is filtered though 26-27 G needle while the current partially delipidized adipose matrix is not passed through such tiny needles, instead, is passed through 20-22 G needles.

ii. Preparation of Human and Rat Adipose-Derived Mesenchymal Stem Cells (hMSCs and rMSCs).
a. Cell Expansion.

Cryopreserved hMSCs and rMSCs were obtained from commercial sources. Vials with cells were thawed and cultured in DMEM with 10% FBS or low serum according to the manufacturer's protocols. Cells were passaged at 80-90% confluency.

b. Adipogenic Differentiation:

Culture expanded MSCs were plated in 75 or 175 cm² culture flasks and in wells of a 24-well plate. When cells formed a monolayer DMEM was replaced by an adipogenic differentiation medium. Cells in DMEM-10% FBS served as a control. Medium was changed every 3-4 days. Cells were cultured for 7-10 days with microscopic assessment for the presence of cells with accumulated lipid vacuoles in the cytoplasm. To confirm adipogenic differentiation, cells in a 24-well plate were fixed in 4% formaldehyde and stained with Oil Red O according to a standard protocol. Photographs were taken. As an additional confirmation of adipogenic differentiation, culture supernatants were collected and tested for the presence of adiponectin, a marker of adipogenic differentiation.

iii. Preparation of rMSC and hMSC Formulations: MSC Seeding the Devitalized and Partially Delipidized Adipose Matrix
a. MSCs Seeding on Adipose Matrix:

Culture expanded undifferentiated and 7-10 days adipo-differentiated hMSCs and rMSCs were harvested from culture flasks and seeded on devitalized and partially delipidized adipose matrix. Rat MSCs were seeded on the rat matrix, and hMSCs were seeded on the human matrix at 0.5-10 mil cells per 1 mL adipose matrix in 5-15 mL DMEM-10% FBS in 50 mL tubes. Tubes were incubated overnight at 37° ° C. on the rotator. After overnight incubation, tubes with matrix alone, matrix seeded with undifferentiated MSCs and matrix seeded with adipo-differentiated MSCs were incubated in the CO₂ incubator for 1 to 4 weeks. Medium was changed every 3-4 days. For the use in animal studies, rMSC formulations after overnight cell seeding were transferred into syringes, and the rMSC formulation was injected into animals on the same day.

b. In Vitro Testing of rMSC and hMSC Formulations.

Cell attachment and viability of seeded cells on the matrix were evaluated microscopically at different time points by staining with Calcein/Propidium Iodine (PI) or Acridine Orange (AO)/PI fluorescent dyes or by using the ATP metabolic assay according to the standard protocols. Briefly, ˜20 μL of MSC formulations were mixed with 20 μL of AO/PI or Calcein/PI. Microscopic evaluation of AO/PI and Calcein/PI-stained samples was performed after 5 min and 20 min of staining at room temperature (RT), respectively. For the ATP assay, samples (50 μL/well) in 96-well plates were mixed with 50 μL/well DMEM-10% FBS and 100 μL/well the CellTiter-Glo 2.0 assay reagent. The plate was incubated for 20 min at RT in a dark place followed by the plate reading using a Luminometer.

Detection of adiponectin secreted by rMSC and hMSC formulations in vitro. Tissue culture supernatants were collected at different time points of MSC formulation in vitro culturing and tested for rat or human adiponectin by rat adiponectin ELISA (R&D Systems) or by human adiponectin Luminex kit (Thermofisher) according to the manufacturer's protocols.

### 2. Results

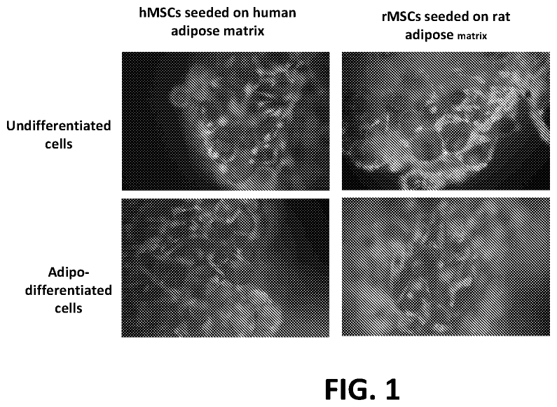

FIG. 1 shows fluorescent microscopic images of Calcein-stained undifferentiated and adipo-differentiated hMSCs and rMSCs one week after cells were seeded on the devitalized and partially delipidized human or rat adipose matrix. Staining showed that fibroblast shaped hMSCs and rMSCs remained viable and attached to the adipose matrix. Staining was performed once a week for 4 weeks. Images on FIG. 1 show viable cells attached to the matrix after 1 week of cell seeding. Cells remained viable and attached to the matrix for the entire duration of the study (4 weeks, data not shown).

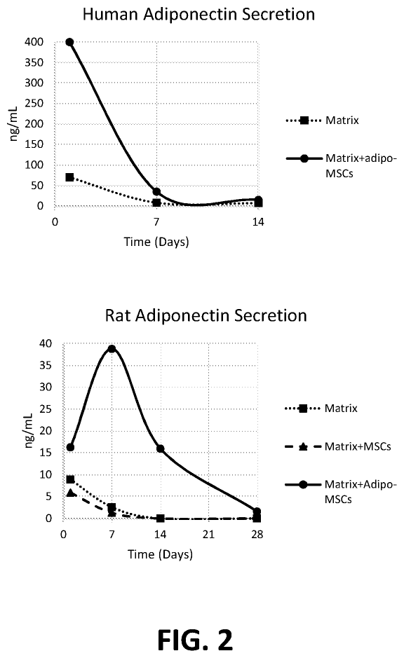

FIG. 2 shows secretion of adiponectin by hMSC and rMSC formulations in vitro in tissue culture medium over time. The graph on the left shows that the hMSC formulation comprised of adipo-differentiated hMSCs seeded on human adipose matrix secreted 8-to-10-fold higher levels of human adiponectin as compared to the matrix alone one day after the cell seeding. However, the amount of adiponectin sharply dropped during next 7 days. This result shows that hMSCs after seeding on the adipose matrix are undergoing de-differentiation when cultured in DMEM. Adiponectin secretion by the rMSC formulation was increasing and reached the peak 7 days after cell seeding on the matrix (right graph) followed by a decrease during next 3 weeks. Similar to the hMSC formulation, the rMSC formulation secreted higher levels of adiponectin as compared to the matrix alone or matrix seeded with undifferentiated rMSCs.

### B. Example 2. Rat MSC Formulation Testing In Vivo in Nude Mice

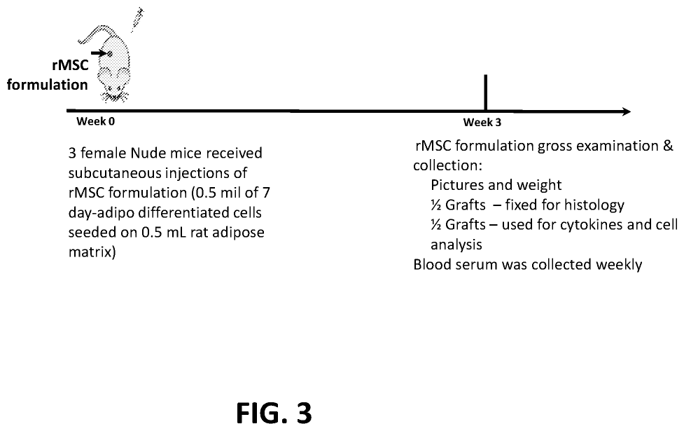

Study design: This study was conducted using three 8 weeks old female athymic nude mice. The duration of the study was 3 weeks. After undergoing general anesthesia, mice received subcutaneous injections of the rMSC formulation consisting of 0.5 million of 7 days adipo-differentiated rMSCs seeded on 0.5 mL devitalized and partially delipidized rat adipose matrix. The rMSC formulation was injected subcutaneously on the back of each mouse via 20 G needles. Pain was controlled by pain medication, Buprenorphine, at 0.05-0.1 mg/kg SQ, q8-12 hr for 2-3 days post injection. Inflammation was controlled by Rimadyl (Carprofen) at 5 mg/kg SQ, q24 hr. On the day of the injection a baseline blood serum sample was collected from all mice prior to the rMSC formulation injection. Remaining post-injection rMSC formulations were fixed in 10% formalin for histological evaluation. At week 1, 2 and 3 post-injection blood serum samples were collected. Body weights were measured weekly. At the end of the study, mice were sacrificed. The skin on the back of each mouse was cut, and the rMSC formulation grafts were gross examined, photographed and dissected out. Each graft was weighted, and then divided into 2 halves. One half of the graft from each animal was fixed in formalin for histological analysis, and another half of the graft from each animal was collected in a tube with DMEM medium for cytokine and cell analyses. Fixed samples were sent to a histology lab. FIG. 3 shows the study design.

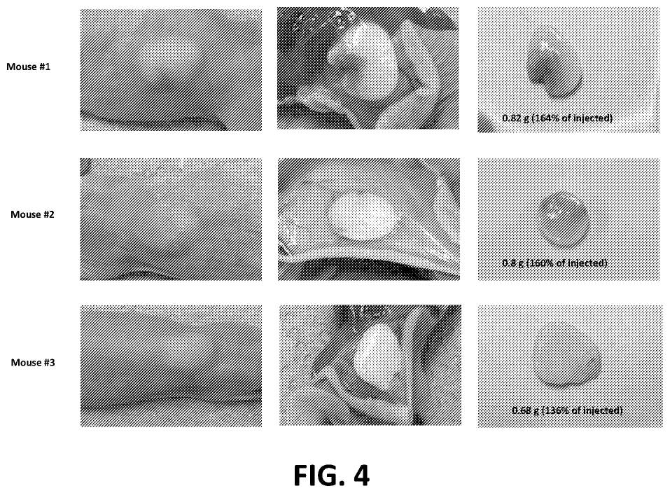

FIG. 4 shows visual appearance and weight for rMSC formulation excised from nude mice 3 weeks post-injection. After 3 weeks, each rMSC formulation weighed more than 0.5 g of the injected material. The increase was 64%, 60% and 36% for the rMSC formulation excised from mouse #1, #2 and #3, respectively (FIG. 4).

Histological evaluation: fixed rMSC formulation samples were stained by hematoxylin and eosin (H&E). Histological analysis included evaluation of tissue integrity and remodeling of H&E stained rMSC formulation sections. Five different fields per each section were examined and graded. Parameters included: the presence of necrosis; inflammation (as evidenced by host cell infiltration of the grafts); and the presence of fibrosis. Each of these parameters was graded on a semiquantitative scale ranging from 0 to 4 by evaluation of the relative presence of each of the histologic parameters in the slide under examination, as follows: 0 (absence), 1 (1-25% tissue involved), 2 (26-50% tissue involved), 3 (51-75% tissue involved), 4 (76-100% involved). H&E-stained rMSC formulation materials served as a time “zero” control.

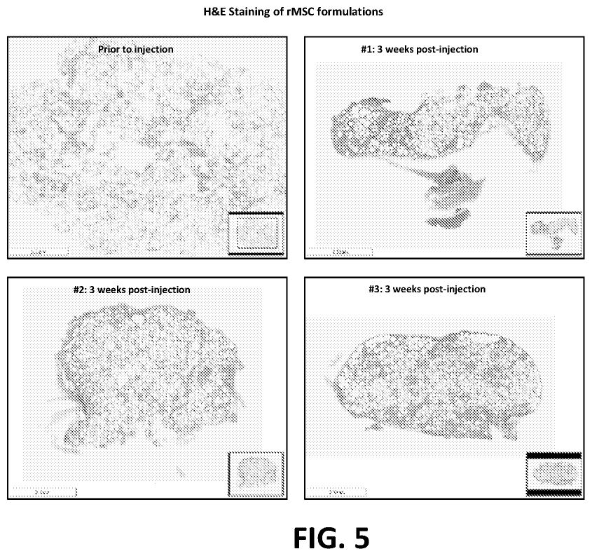

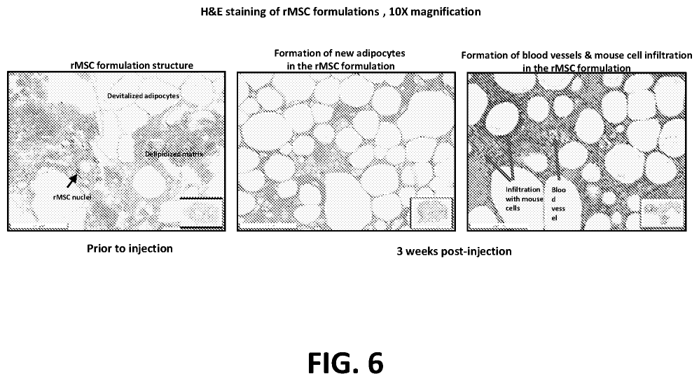

FIGS. 5 and 6 show H&E-stained sections of the rMSC formulation prior and 3 weeks post-injection at low and higher magnifications, respectively. Histologically, prior to injection rMSC formulation contained areas consistent with fibrous tissue, which represents delipidized adipose matrix, and devitalized mature adipocytes with sparse rMSCs attached to the delipidized matrix (FIG. 6). Three weeks post-injection the rMSC formulation was undergoing remodeling: 1. Mouse inflammatory cells migrate into the rMSC formulation, and macrophages started uptaking lipids released from dead rat adipocytes; 2. At the same time, formation of new adipocytes and new blood vessels was ongoing in the rMSC formulations. The described remodeling is supported by histological scores for the rMSC formulation prior vs 3 weeks post-injection (Table 1).

Table 1 shows mean histological scores for the rMSC formulations excised from nude mice 3 weeks post-injection. The scores for the rMSC formulation prior to injection are presented for comparison.

rMSC Formulation,
Histological Scores (Mean ± SD)

| Histological | Prior to | 3 Weeks post- |
|---|---|---|
| Parameters: | injection | injection |
| Integrity | 0.00 | 2.67 ± 0.58 |
| Oil Vacuoles | 3.00 | 3.00 ± 0.00 |
| Fibrous Tissue | 3.00 | 3.00 ± 0.00 |
| Inflammation | 0.00 | 0.67 ± 1.15 |

Detection of rat adiponectin in serum of nude mice post-injection of the rMSC formulation: Mouse serum was collected at baseline (prior to rMSC formulation injection) and at weeks 1, 2 and 3 post-injection. Serum was tested at 50% concentration for the presence of rat adiponectin using ELISA (R&D Systems) according to manufacturer's protocol. Mouse serum prior to rMSC injection served as a negative baseline control. Results showed that at all study time points rat adiponectin was not detectable in mouse serum after rMSC formulation injection (data not shown). These results indicate that subcutaneously injected rMSC formulation has no systemic effect.

Analysis of rMSC formulation excised from nude mice. Detection of viable cells in the rMSC formulation: a small slice from each excised rMSC formulation was stained with AO/PI and then examined microscopically. Detection of rat adiponectin secreted by the excised rMSC formulations in vitro. Collected tissues were cut in 2 halves. One half was placed in 1 mL DMEM-10% FBS for 48 h. Then, culture supernatants were collected and analyzed for the presence of rat adiponectin by ELISA. Isolation and culturing cells from the excised rMSC formulations. Another half of collected grafts was used for cell isolation. Cells were isolated from 3 samples of the excised rMSC formulation by digestion with collagenase type 2. Grafts were cut into small pieces by scissors, and then digested 45 min at 37° C. in 15 mL tubes in 5 mL DMEM+0.25 mL 2% collagenase. Digested tissues were filtered via a 300 nM nylon filters and centrifuged. Cells were plated in 3 mL complete DMEM-10% FBS in wells of a 6-well plate. Culture expanded cells were used in further experiments. Detection of rat mesenchymal stem cells in the cell population isolated from the excised rMSC formulations using adipogenic differentiation: Culture expanded cells were plated in a 24-well plate. When cells formed a monolayer, DMEM-10% FBS was replaced by the adipogenic differentiation medium. Cells in DMEM-10% FBS served as a control. Medium was changed every 3-4 days. Cells were cultured for 3 weeks with microscopic assessment for the presence of cells with accumulated lipid vacuoles in the cytoplasm. After 3 weeks, cells were fixed in 4% formaldehyde and stained with Oil Red O for detection of adipo-differentiated cells. Photographs were taken. Culture supernatants from wells with the control and the adipo-differentiated cells were collected and tested for the presence of rat adiponectin by ELISA.

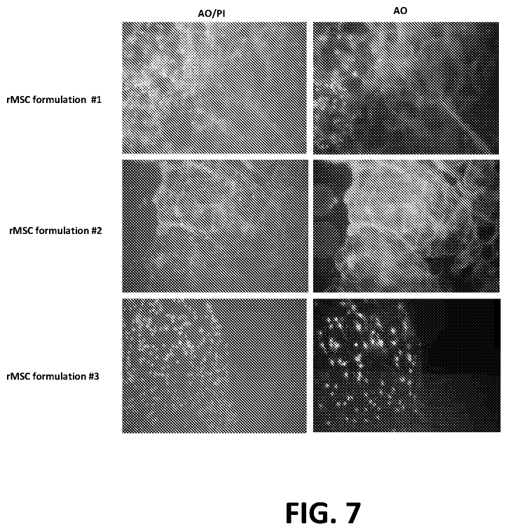

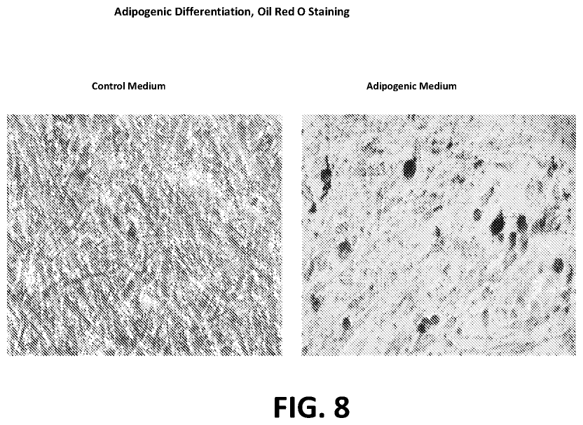

FIG. 7 shows that excised rMSC formulations 3-weeks post-injection contained viable cells. Viable cells of fibroblast morphology were isolated from the rMSC formulation and successfully cultured ex vivo. Adipogenic differentiation of culture expanded cells demonstrated that some of these cells are mesenchymal stem cells with a potential for differentiation into adipocytes (FIG. 8). However, rat MSCs were not detected in the population of cells isolated from the rMSC formulations: there were no rat adiponectin detected in culture supernatants collected from wells with adipo-differentiated cells. This result indicated that rMSCs were replaced in the formulation by mouse cells. After 3 weeks, only residual rat adiponectin was released from the rMSC formulation. Results indicate that the source of residual rat adiponectin is rat adipose matrix. Adiponectin levels were ˜150-fold below the level of rat adiponectin secreted by the rMSC formulation prior to the injection (Table 2). This result is in line with the observed decrease in adiponectin secretion by the rMSC formulation in vitro overtime (FIG. 2, bottom graph).

Table 2 shows amount of rat adiponectin released by rMSC formulation in culture medium.

| | Rat adiponectin |
|---|---|
| | (in pg/24 h/0.5 mL |
| Sample | of matrix) |
| rMSC formulation prior to | 33,000 |
| injection in nude mice | |
| rMSC formulation excised from | 197 +/− 65 |
| mice 3 weeks post-injection | (Mean +/− SD, |
| | n = 3) |

In summary, rMSC formulation in vitro and in vivo testing include: Adipo-differentiated MSCs seeded on the adipose matrix survive at least 4 weeks in vitro and represent the main source of adipokines secreted by the formulation; 3 weeks after injection of rMSC formulation into nude mice, the rat matrix was populated by mouse cells and became larger in size. However, viable rMSCs were not detected, and the amount of adipokines secreted by the rMSC formulation was negligible 3 weeks post-injection; Remodeling of the rMSC formulation in vivo included the formation of new mouse-derived adipocytes and blood vessels with a simultaneous resorption of rat dead adipocytes in the rat adipose matrix.

### C. Example 3. Development and Treatment of Non-Alcoholic SteatoHepatitis (NASH) by the rMSC Formulation in Obese Fa/Fa Zucker Rats

Study design: This study had two parts: part 1—NASH model development; and part 2—treatment of NASH in rats with the rMSC formulation.

Part 1. Model development. Ten rats were fed HFC-F diet. At weeks 1 (Day 7), 4 (Day 25), 8 (Day 55), and 15 (Day 105) one rat was euthanized, gross examination of adipose tissue and liver was performed, and liver weight was recorded. The terminal blood, liver and adipose tissues were collected for biochemical and histological (liver only) analysis. The development of NASH was evaluated histologically.

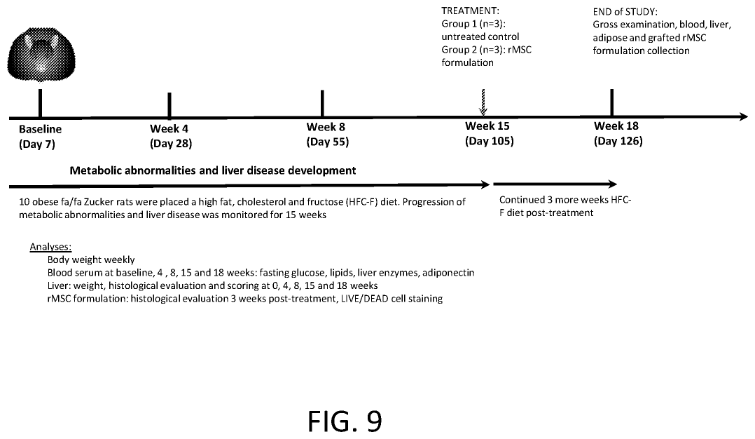

Part 2. Treatment of NASH in rats with the rMSC formulation. On day 105 on HFC-F diet, 6 rats were divided in 2 groups: untreated control group 1 (n=3) and treated group 2 (n=3). Group 1 received no treatment. Group 2 received one injection of the rMSCs formulation (rMSCs in rat adipose matrix) in 4 points in abdominal subcutaneous fat and one injection of the rMSC formulation subcutaneously on the dorsum. All animals continued to stay on the HFC-F diet for additional 3 weeks. On day 126, rats were euthanized, gross examination and liver weighing were performed. The terminal blood, liver and adipose tissues were collected for biochemical and histological (liver only) analysis. The injected rMSC formulation from the abdominal fat area was identified and dissected out. One half of the rMSC formulation was collected in tubes with medium for cellular and biochemical evaluation, and another half of the rMSC formulation was placed in 10% formalin for histological analysis. Animals were maintained for 126 days with weekly body weights. The schematic presentation of the study design is shown in FIG. 9.

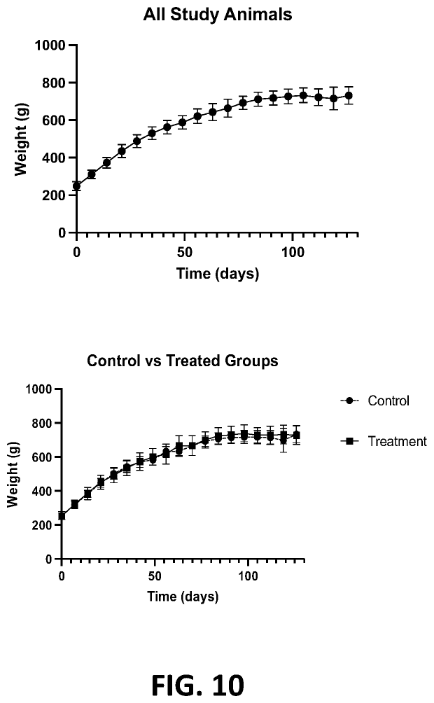

Body weights, gross examination and liver weights. Body weights were measured weekly until the end of the study. Data shows that all rats were gaining weight as expected. Table 3 and FIG. 10 summarize the descriptive statistics of the body weight change from Day 0 to Day 126. The right graph on FIG. 10 shows that there were no differences in body weight between control and treated groups.

| | Body Weight (g) | | | | | | | | |
|---|---|---|---|---|---|---|---|---|---|
| Study | All Study Animals | | | Control Group | | | Treatment Group | | |
| Day | Mean | SD | N | Mean | SF | N | Mean | SD | N |
| 0 | 249 | 24 | 10 | 255 | 24 | 3 | 251 | 20 | 3 |
| 7 | 311 | 23 | 9 | 319 | 21 | 3 | 322 | 25 | 3 |
| 14 | 373 | 28 | 9 | 383 | 21 | 3 | 385 | 37 | 3 |
| 21 | 435 | 35 | 9 | 448 | 28 | 3 | 452 | 42 | 3 |
| 28 | 488 | 34 | 8 | 501 | 32 | 3 | 493 | 45 | 3 |
| 35 | 530 | 34 | 8 | 542 | 33 | 3 | 535 | 45 | 3 |
| 42 | 563 | 36 | 8 | 570 | 32 | 3 | 573 | 52 | 3 |
| 49 | 588 | 35 | 8 | 583 | 29 | 3 | 600 | 50 | 3 |
| 56 | 621 | 39 | 7 | 633 | 29 | 3 | 617 | 58 | 3 |
| 63 | 643 | 45 | 7 | 633 | 29 | 3 | 667 | 58 | 3 |
| 70 | 664 | 48 | 7 | 667 | 58 | 3 | 667 | 58 | 3 |
| 77 | 692 | 35 | 7 | 693 | 31 | 3 | 700 | 48 | 3 |
| 84 | 711 | 38 | 7 | 707 | 34 | 3 | 724 | 49 | 3 |
| 91 | 718 | 38 | 7 | 714 | 35 | 3 | 731 | 50 | 3 |
| 98 | 726 | 39 | 7 | 717 | 38 | 3 | 739 | 50 | 3 |
| 105 | 732 | 39 | 6 | 717 | 39 | 3 | 728 | 46 | 3 |
| 112 | 722 | 44 | 6 | 714 | 42 | 3 | 729 | 54 | 3 |
| 119 | 715 | 60 | 6 | 698 | 71 | 3 | 732 | 56 | 3 |
| 126 | 731 | 47 | 5 | 734 | 52 | 2 | 729 | 55 | 3 |

Animals were euthanized according to the protocol without complication. Tissues were collected as described in the protocol. Noticeable increase in the abdominal fat overtime was observed in the rats fed the HFC-F diet. There were no visual differences between groups and between animals in the group (data not shown).

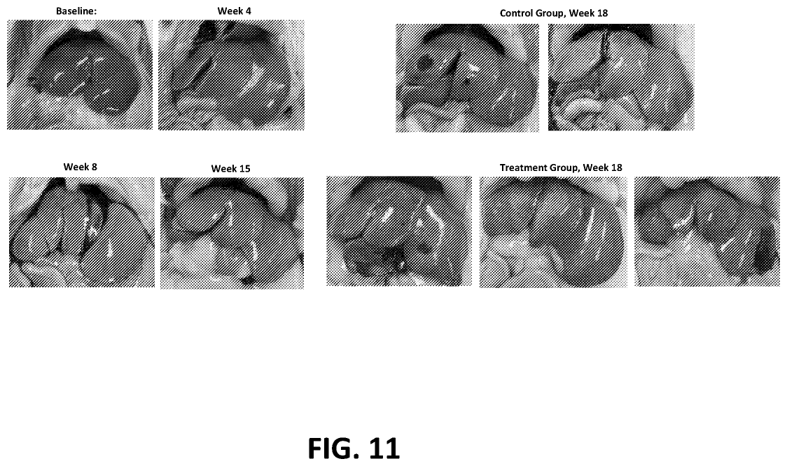

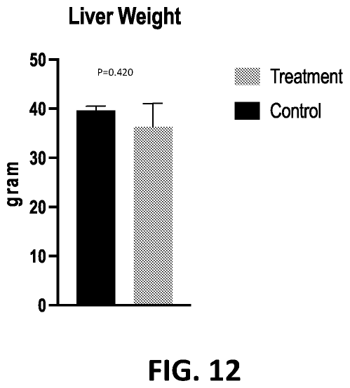

FIG. 11 shows changes in liver size and color from the baseline (day 7) till week 18 (Day 126). In comparison to the baseline (day 7), which shows normal liver appearance, all livers at other time points were larger and pale in color (greyish), which is a sign of the fatty liver disease with an increase in the days on HFC-F high diet (FIG. 11). There were no visual differences in liver appearance between groups and between animals in the group (FIG. 11). Liver weights are summarized in Table 4. One rat from the control group was excluded from the table. This animal died on day 119 of the study. Table 4 shows that liver weights were increasing with time during when the animals were fed the HFC-F diet. The comparison of liver weights between control and treated rats shows smaller livers for the treatment group; however, the difference was not significant (FIG. 12).

Table 4 summarizes liver weight for each animal during the study.

| | Animal | Group | Liver weight |
|---|---|---|---|
| Study Timepoint | Number | Assignment | (grams) |
| Week 18 (day 126) | 1 | control | 39.1 |
| Week 18 (day 126) | 3 | control | 40.3 |
| Week 18 (day 126) | 4 | treatment | 31.5 |
| Week 18 (day 126) | 5 | treatment | 41 |
| Week 18 (day 126) | 6 | treatment | 36.6 |
| Week 15 (day 105) | 7 | control | 35.4 |
| Week 8 (day 55) | 8 | control | 28.5 |
| Week 4 (day 28) | 9 | control | 25.9 |
| Baseline (day 7) | 10 | control | 17.4 |

Serum samples analysis. Blood was collected from 4 study animals on Days 7 (baseline), 28, 55 and 105 (one animal per time point) and from all five remaining study animals on Day 126. Blood was collected into 2 ml serum separator tubes and processed to serum. Serum was aliquoted into two (2) tubes at approximately equal volumes for each animal and stored at −80° ° C. prior to analysis. Serum samples were used for blood biochemistry analysis including fasting glucose, lipids and liver enzymes and for serum adiponectin detection.

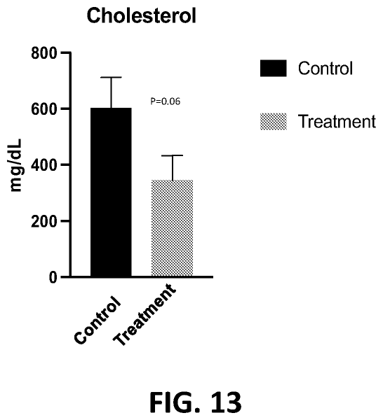

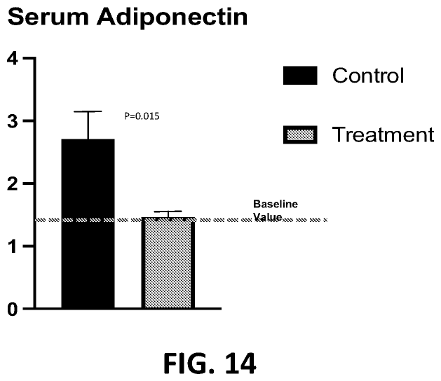

Table 5 summarizes serum liver enzymes, glucose, lipids and adiponectin results for each study animal. One animal from the control group (#2) died on Day 119, and blood was not collected. Values for liver enzymes and blood glucose show that the main spike with highest numbers were observed after 4 weeks (Day 28) on HFC-F diet. After 4 weeks on HFC-F diet values for liver enzymes were decreasing overtime, which was not expected, but can be explained by increase in adipose tissue as a compensatory mechanism protecting animals from ectopic lipid accumulation. Cholesterol and triglyceride levels were increasing over time as expected. Although the difference in cholesterol values on Day 126 between control and treated animals did not reach statistical significance, the result showed a clear trend. Treatment of animals on HFC-F diet with the rMSC formulation resulted in cholesterol decrease (FIG. 13). Serum adiponectin in the control group was significantly higher as compared to the level of serum adiponectin in the treatment group (FIG. 14).

Table 5 shows blood serum test results for each animal.

| | Study Time point: | | | | | | | | | |
|---|---|---|---|---|---|---|---|---|---|---|
| | Baseline | Week 4 | Week 8 | Week 15 | Week 18 Treatment | | | Week 18: | | |
| | (Day 7) | (Day 28) | (Day 55) | (Day 105) | Group | | | Control Group | | Normal |
| Animal # | 10 | 9 | 8 | 7 | 6 | 5 | 4 | 3 | 1 | Range |
| AST | 167 | 326 | 356 | 156 | 190 | 100 | 94 | 97 | 100 | 10-45 |
| | | | | | | | | | | IU/L |
| ALT | 107 | 127 | 117 | 90 | 68 | 61 | 58 | 37 | 75 | 10-35 |
| | | | | | | | | | | IU/L |
| Alk | 269 | 546 | 186 | 327 | 275 | 154 | 141 | 150 | 227 | 15-45 |
| phosphatase | | | | | | | | | | IU/L |
| Glucose | 199 | 260 | 193 | 168 | 117 | 159 | 136 | 133 | 145 | 60-125 |
| | | | | | | | | | | mg/dL |
| Cholesterol | 118 | 260 | 276 | 408 | 325 | 270 | 443 | 681 | 525 | 50-250 |
| | | | | | | | | | | mg/dL |
| Triglycerides | 311 | 295 | 400 | 741 | 285 | 646 | 837 | 855 | 1012 | <150 |
| | | | | | | | | | | mg/dL |
| Adiponectin | 1.457 | 0.819 | 0.814 | 1.446 | 1.544 | 1.361 | 1.480 | 2.394 | 3.022 | Baseline, |
| | | | | | | | | | | ocg/mL |

Histological evaluation: fixed rMSC formulation and liver samples were stained by hematoxylin and eosin (H&E) and Masson's trichrome (MT). Histological analysis included evaluation of tissue integrity and remodeling of H&E stained rMSC formulation sections. Five different fields per each section were evaluated and graded. The parameters included: the presence of necrosis; inflammation (as evidenced by host cell infiltration of the grafts); and the presence of fibrosis. Each of these parameters was graded on a semiquantitative scale ranging from 0 to 4 by evaluation of the relative presence of each of the histologic parameters in the slide under examination, as follows: 0 (absence), 1 (1-25% tissue involved), 2 (26-50% tissue involved), 3 (51-75% tissue involved), 4 (76-100% involved). H&E-stained rMSC formulation materials served as a time “zero” control. Liver sections were scored for the degree of steatosis as the percentage of hepatocytes containing lipid droplets. Inflammation was evaluated as the sum of scores (score 0-6) for acinar inflammation (score 0-3) and portal inflammation (score 0-3). Fibrosis was graded from 0 (absent) to 4 (1, perisinusoidal/pericellular fibrosis; 2, periportal fibrosis; 3, bridging fibrosis; 4, cirrhosis).

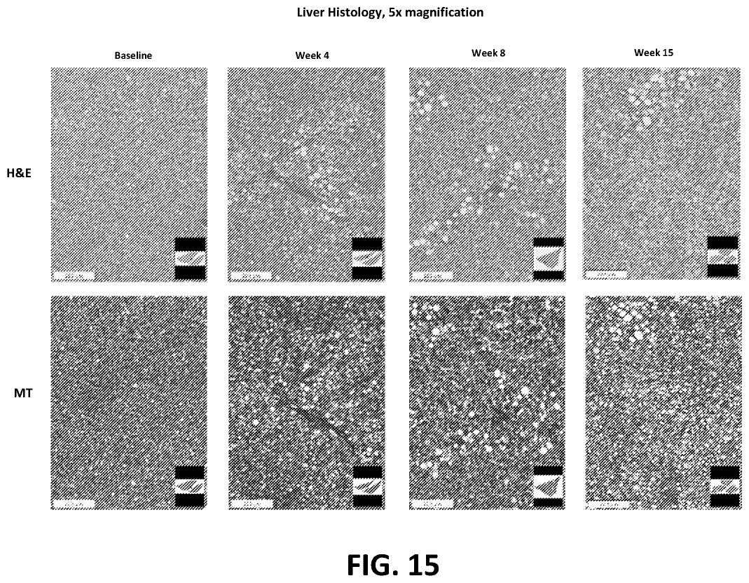

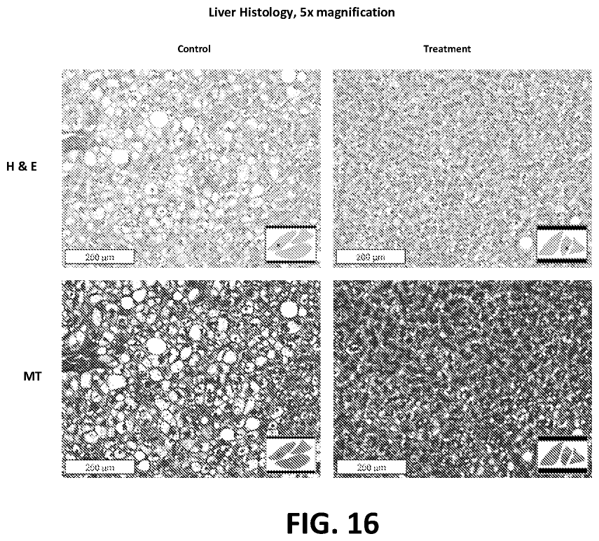

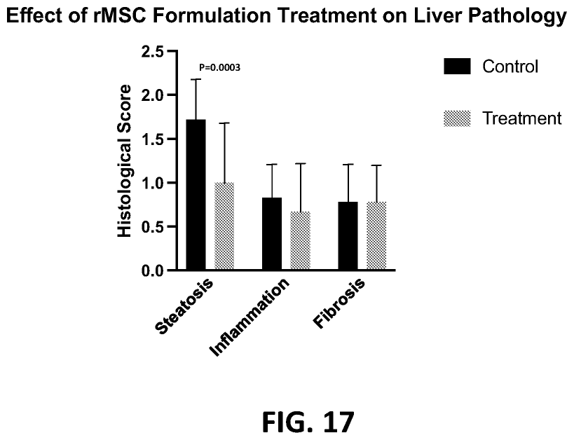

Fixed liver tissues were sent to a histology lab for H&E and MT staining. Stained sections were analyzed by a certified pathologist. FIG. 15 shows representative images of liver sections from the baseline (day 7) till week 15 (Day 105). These 4 animals were used to monitor development of fatty liver and NASH. Images show increase in liver steatosis, however, inflammation and fibrosis in the liver remained minimal at each timepoint. FIG. 16 shows representative images of liver sections for control and treated animals at the end of the study (Day 126). The treatment resulted in decrease of hepatocytes containing large lipid droplets (macrovesicular steatosis), which is a positive outcome (FIG. 16, left images vs right images). Steatosis, inflammation and fibrosis scores for control and treated animal liver sections are shown in FIG. 17. There was a significant difference in the liver steatosis score between the control and treatment groups; however, the difference in inflammation and fibrosis scores was not significant between groups (FIG. 17). The decrease in liver steatosis score in the treated rats correlated with decrease in serum cholesterol (FIG. 13).

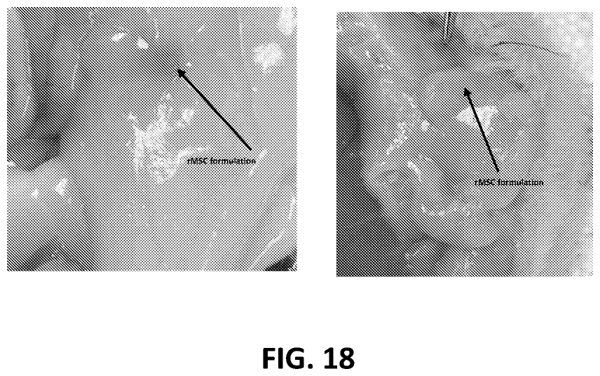

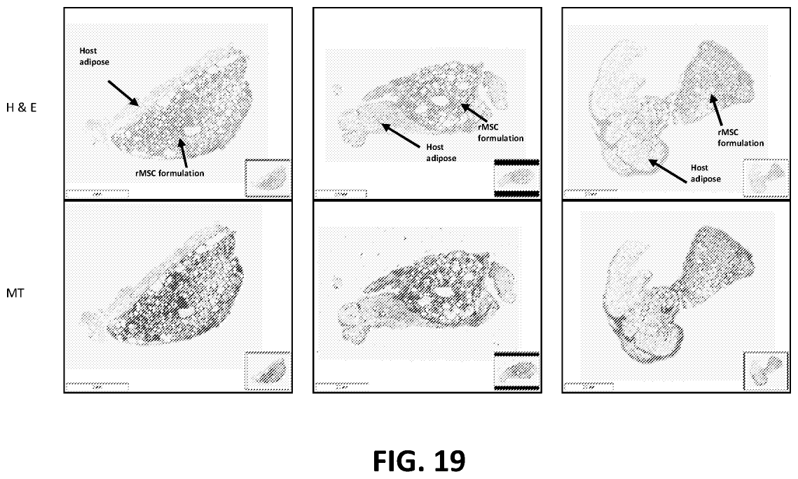

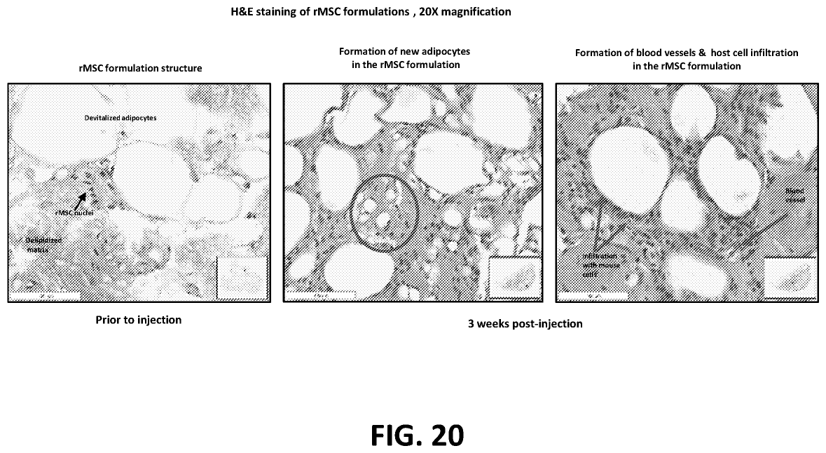

The abdominal subcutaneous adipose area of the rMSC formulation injections was examined. FIG. 18 shows visual appearance of the rMSC formulation at the subcutaneous abdominal adipose injection sites 3 weeks post-injection. rMSC formulations were excised from 3 treated animals for histological and cell analysis. FIG. 19 shows histological appearance of the excised rMSC formulation 3 weeks post-injection in rats at low magnification, and Table 6 summarizes mean and SD scores vs the scores for the rMSC formulation prior to the injection. FIG. 20 shows H&E-stained sections of the rMSC formulation prior and 3 weeks post-injection at high magnification. Histologically, prior to injection the rMSC formulation contains areas consistent with fibrous tissue, which represent delipidized adipose matrix and devitalized mature adipocytes with sparse rMSCs attached to delipidized matrix (FIG. 20). After 3 weeks post-injection the rMSC formulation was undergoing remodeling: 1. Zucker rat inflammatory cells migrates into the rMSC formulation and macrophages started uptaking lipids released from dead rat adipocytes; 2. At the same time, formation of new adipocytes and new blood vessels was ongoing in the rMSC formulations. The described remodeling is supported by histological scores for the rMSC formulation prior vs 3 weeks post-injection (Table 6). The remodeling and histological scores for the rMSC formulation injected in rats (FIG. 20, Table 6) are similar to those recorded for the rMSC formulation injected in nude mice (FIG. 6, Table 1).

Table 6 shows mean histological scores for the rMSC formulations excised from Zucker rats 3 weeks post-injection. The scores for the rMSC formulation prior to injection are presented for comparison.

rMSC Formulation,
Histological Scores (Mean ± SD)

| | Prior to | 3 Weeks |
|---|---|---|
| Historical Parameters: | injection (Control) | post-injection |
| Integrity | 0.00 | 1.00 ± 1.00 |
| Oil Vacuoles | 3.00 | 3.00 ± 0.00 |
| Fibrous Tissue | 3.00 | 1.67 ± 0.58 |
| Inflammation | 0.00 | 1.33 ± 0.58 |

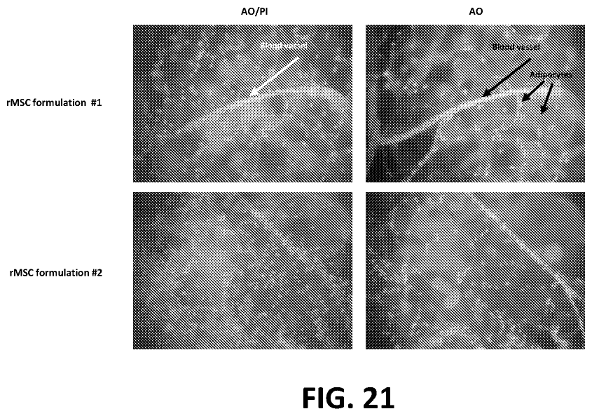

Detection of viable cells in the rMSC formulation: a small slice from each excised rMSC formulation was stained with AO/PI, and then examined microscopically. FIG. 21 shows that excised rMSC formulations 3 weeks post-injection contain viable cells including newly formed adipocytes and newly formed blood vessels inside the rMSC formulations. These adipocytes and blood vessels were not present in the rMSC formulation; they were formed during 3 weeks in vivo by the host cells.

Summary. The purpose of this study was to develop a rat model of non-alcoholic steatohepatitis (NASH) and to evaluate effects of rMSC formulation on metabolic parameters and NASH in an obese fa/fa Zucker rat model. Ten six weeks old male obese fa/fa Zucker rats were on a high fat & high cholesterol & high fructose (HFC-F) diet for the duration of the study. Four animals were used to monitor progress of the liver disease development from the baseline (day 7) till week 15 (day 105) of the study. The development of NASH was evaluated histologically. Liver histology showed increase in liver steatosis; however, inflammation and fibrosis in the liver were minimal for each timepoint. Blood was also collected from each animal for analysis of fasting glucose, liver enzymes, lipids and adiponectin. Cholesterol and triglyceride are significantly increasing over time. The increase in cholesterol and triglyceride correlates with the increase in liver steatosis. The obese fa/fa Zucker rats on HFC-F diet develop fatty liver disease with early NASH and metabolic abnormalities such as diabetes and hyperlipidemia. The developed model is appropriate for evaluation of therapies for metabolic and liver diseases.

On day 105 of the study, six rats were divided into two groups of three animals each: Group 1 (untreated control) and Group 2 (treatment) received 6×10⁶ rMSC in 2 mL rat adipose matrix in 4 points in abdominal subcutaneous fat and in 1 point subcutaneously on the dorsum (0.4 mL of ˜1.2×10⁶ rMSCs per point). Treatment with the rMSC formulation decreased liver steatosis and hyperlipidemia in Zucker obese rat that were fed by the HFC-F diet. The study results support the hypothesis that the rMSC formulation has a therapeutic effect on fatty liver disease and metabolic abnormalities.

### D. Example 4

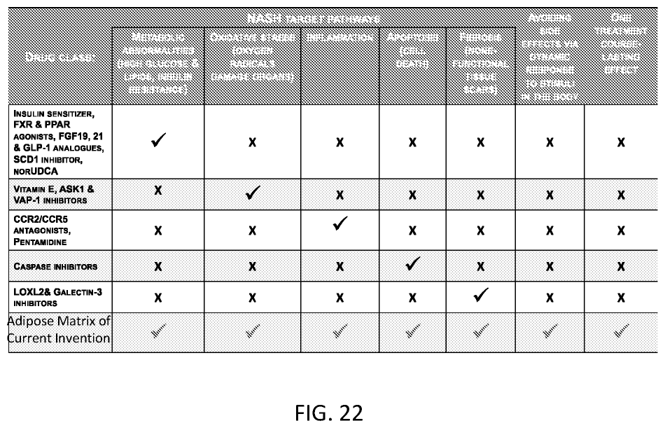

Over the past 40 years, the effort to develop drugs for nonalcoholic steatohepatitis (NASH) has not been successful; there are no approved NASH therapies. To address this unmet medical need, the disclosed adipose matrix and compositions comprising the adipose matrix have been developed as a therapy for NASH. As described herein, the composition is comprised of mesenchymal stem cells (MSCs) seeded on a partially delipidized adipose matrix. Experimental data show that transplantation of white or brown adipose from normal mice to obese mice restores disrupted metabolism that results in weight loss, reduction of insulin resistance and liver steatosis (Ablamunits et al., J Endocrin, 2012; 212: 41; Liu et al., Endocrinology, 2015; 156: 2416). However, this approach is not feasible for the treatment of patients due to a requirement for immunosuppression to prevent rejection. Recent studies report that injections of MSCs in rodents fed a high fat diet can improve metabolism and liver function (Li et al., Stem Cells Int, 2019:8628027; Domingues et al., Stem Cell Res Ther, 2019; 10: 280). Based on these data, it was hypothesized that a combination of MSCs and an adipose matrix can have therapeutic effects similar to the adipose transplant, but without its limitations. The adipose matrix described herein is an essential component that serves as a MSC carrier and provides a cell-friendly environment to support the functionality of transplanted MSCs and host cells. Paracrine secretion of growth factors and cytokines and differentiation in adipocytes are two mechanisms by which MSCs can mediate the therapeutic effects. The strategy for NASH therapy is to target disrupted metabolism in adipose tissue, which is the root cause of NASH. It has been established that the primary mechanism that leads to NASH is lipotoxic liver injury due to the failure of the adipose tissue to store an excess of lipids. An imbalance between lipid synthesis and breakdown results in high levels of fatty acids being released from the adipose tissue. Released fatty acids trigger insulin resistance, hyperglycemia, and hyperlipidemia, which damage blood vessels and organs including the liver (Rutkowski et al., J Cell Biol, 2015; 208: 501; Jönsson et al., Biochem J, 2019; 476: 2883). By targeting disrupted metabolism in the adipose tissue, the disclosed compositions can be effective not only for NASH but also for other metabolic abnormalities (e.g., insulin resistance, dyslipidemia). The disclosed composition comprising partially delipidized and devitalized adipose matrix and MSCs advantages vs. pharmaceutical and biologic drugs include the potential to address NASH complexity by targeting multiple pathogenic pathways (FIG. 22) and lack of any toxicity. Hepatocyte therapy can provide an alternative solution to liver transplant for patients with liver failure of all causes including NASH; however, it is not targeting NASH pathogenesis. The disclosed compositions are designed to compensate for the lost functionality of patients' adipose tissue. Other MSC technologies are not addressing dysfunctional adipose tissue, which is the trigger for NASH.

One conclusion from these experiments is that the disclosed composition decreases liver pathology (FIG. 16) in a diet-induced obese Zucker rat NASH model. Matrix-free suspension of MSCs showed no therapeutic effect. Ongoing adipogenesis with the mitochondrial uncoupling protein 1 (UCP1) brown adipocyte marker expression and the presence of M2 macrophages were detected in the implanted BRC001 in vivo. This finding together with literature data showing protective effects of M2 macrophages from obesity (Feng et al., Cell Mol Immunol, 2018; 15:493; Su et al., Sci Rep, 2018; 8:4607) indicate that M2 host macrophages and newly formed adipocytes can represent a mechanism of action. A summary of completed studies with key outcomes is presented in Table 7 BRC001 is partially deplipidized and devitalized adipose matrix plus MSCs.

TABLE 7

| Studies | Outcomes |
|---|---|
| BRC001 | BRC001 small scale manufacturing process |
| process | has been developed. |
| development | BRC001 cryopreservation and storage at |
| | −80° C. is feasible. |
| BRC001 | Lipid content: 50-70% |
| Characterization | Adipokines: adiponectin level is similar |
| | to native adipose. |
| | Cell Viability: MSCs seeded on adipose |
| | matrix are viable for at least 4 weeks |
| | in vitro. |
| Diet-induced | The model has been developed and it shows |
| NASH model in | early stages of NASH detectable after 4 |
| obese Zucker | weeks on the high fat-cholesterol-fructose |
| rats | diet. Animals also develop hyperglycemia |
| | and hyperlipidemia. |
| Rat BRC001 | Efficacy: BRC001 (2 mL matrix containing |
| (Sprague | 6 mL MSCs) was injected in the subcutaneous |
| Dawley (SD) rat | fat in obese Zucker rats. 3 weeks post- |
| MSCs seeded on | treatment liver pathology scores and |
| the adipose | hyperlipidemia were decreased. |
| matrix) testing | Safety: BRC001 therapy was well tolerated. |
| in the obese | BRC001 fate in vivo: ongoing adipogenesis |
| Zucker rat | with the UCP-1 brown adipocyte marker and |
| NASH model | the presence of host M2 macrophages were |
| (allogeneic rat | detected in the implanted BRC001 matrix 3 |
| BRC001 use) | weeks post-injection. |

### E. Example 5. In Vitro and In Vivo Evaluation of Adipose Matrix Formulations Comprised of Fully and Partially Delipidized Adipose Matrix without and with Adipose-Derived Mesenchymal Stem Cells (MSCs)

Preparation of partially and fully delipidized rat adipose matrix: Subcutaneous abdominal adipose from Zucker rats was washed in PBS, cut with scissors, and then minced with a mezzaluna until the tissue looked like a “baby food puree”. Fibrous pieces were removed.

Partially delipidized adipose matrix: Minced tissue was emulsified by moving between two 10 mL connected syringes. Emulsified adipose was collected in 4×50 mL tubes (volume 200 cc). Tubes were centrifuged at room temperature 15 min at 3000 rpm. Free lipids from the top were removed and most of the PBS underneath the adipose layer was removed. Yield: 16 cc of partially delipidized adipose (started with 100 cc adipose). Partially delipidized adipose was aliquoted in 10 mL syringes and stored at −80° C.

Fully delipidized adipose matrix: Water was added to 2×50 mL tubes of partially delipidized adipose, and these tubes were incubated 30-40 min at RT on a rotator in water. After incubation, adipose was homogenized using an Omni hand homogenizer 5 min at max speed at RT with 5 min 3000 rpm at RT in between. Each time top adipose layer was transferred into a new 50 mL tube, and the pellets of delipidized matrix were combined in one tube. After 4 rounds of tissue homogenization, delipidized adipose was centrifuged in water 10 min 3000 rpm at RT. Water was decanted, and delipidized adipose was transferred in a 10 ml syringe. Yield: ˜4 cc (started volume−100 cc). Delipidized adipose was stored in a syringe at −80° C.

Preparation of therapeutic formulations for treatment of obese Zucker rats: All formulations were prepared on the treatment day of animals using cryopreserved matrix and cells.

Preparation of cells: Thawed and counted culture expanded SD rat adipose derived MSC-GFP: 7 vials×5 mil/vial, P6, cryopreserved in Cryostor. MSC count and viability: 35 million cells, 85.5% viability. Cells were pelleted by centrifugation, resuspended in 2 mL of DMEM and transferred into a 3 mL syringe for mixing with rat partially and fully delipidized adipose matrix.

Fully delipidized matrix formulations (M1 and M1+ cells): Thawed fully delipidized adipose matrix (4 cc matrix), and divided it into 2 portions.

M1 formulation (matrix alone): Added DMEM to make volume 6 mL. M1 formulation contained ˜0.33 cc matrix/mL.

M1+ cells formulation: Added ˜18 mil cells in DMEM to M1 matrix making total volume 6 mL. M1+ cells formulation contained 0.33 cc matrix and 3 mil cells/mL. Cells were mixed with the matrix using two connected syringes.

Partially delipidized matrix formulations (M2 and M2+ cells): Thawed partially delipidized adipose matrix (5.4 cc matrix), and divided it into 2 portions.

M2 formulation (matrix alone): Added DMEM to make volume 6 mL. M1 formulation contained 0.45 cc matrix/mL.

M2+ cells formulation: Added ˜18 mil cells in DMEM to M2 matrix making total volume 6 mL. M2+ cells formulation contained 0.45 cc matrix and 3 mil cells/mL. Cells were mixed with the matrix using two connected syringes.

Characterization of therapeutic formulations: Fully (M1) and partially delipidized (M2) adipose matrices was characterized in vitro for lipid and DNA content.

M1, M1+ cells, M2 and M2+ cells formulations were characterized for adiponectin and VEGF. Conditioned medium (CM) was collected after 48 h incubation of each formulation (˜0.3 cc) in 5 mL DMEM+10% FBS.

Presence of viable cells in the formulations was assessed microscopically after staining with Calcein AM. The structure of the formulations was assessed histologically using H&E-stained sections.

Levels of adiponectin in CMs released from prepared formulations were tested using a rat adiponectin ELISA DUO set (R&D Systems) according to manufacturer's protocol.

Levels of VEGF in CMs released from prepared formulations were tested using a rat VEGF-A ProCarta Simplex kit (ThermoFisher) according to manufacturer's protocol.

Lipid content was measured in M1 and M2 adipose matrix lipid extracts using a triglyceride kit (Cayman) according to manufacturer's protocol.

DNA content measurement in M1 and M2 adipose matrix was performed using a high sensitivity Quant-iT dsDNA kit (Invitrogen) according to manufacturer's protocol.

A summary of therapeutic formulation characterization is presented in Table AAA below.

TABLE 8
Characterization of therapeutic formulations.

| | | DNA, | Adiponectin | | VEGF-A | | | |
|---|---|---|---|---|---|---|---|---|
| | | μg/mg | released | | released | | | |
| | Lipids | wet | from the | | from the | | | |
| | (%) | tissue | formulation | | formulations | | Viable | |
| Formulation | w/w | weight | during 48 h | | during 48 h | | MSCs | Structure |
| #1: M1 | 1% | 0.372 | 42.5 | ng | 12 | pg | None | Histologically, |
| (fully | | | | | | | | all formulations |
| delipidized) | | | | | | | | consisted of |
| #2 | NA | NA | 950 | ng | 10 | pg | Present, | mostly foci of |
| M1 + cells | | | | | | | cells were | collagen fibers. |
| | | | | | | | attached to | |
| | | | | | | | the matrix | |
| #3: | 24% | 0.120 | 93.5 | ng | 31.5 | pg | None | |
| M2 | | | | | | | | |
| (partially | | | | | | | | |
| delipidized) | | | | | | | | |
| #4: | NA | NA | 1380 | ng | 75 | pg | Present, | |
| M2 + cells | | | | | | | cells were | |
| | | | | | | | attached to | |
| | | | | | | | the matrix | |

### Evaluation of Adipose Matrix Formulations for Treatment of Liver Steatohepatitis (NASH) and Metabolic Abnormalities in an Obese Fa/Fa Zucker Rat Model.

Animal study design: The study was conducted using 21 male 8-10 weeks old obese fa/fa Zucker rats. The duration of the study was 9 weeks including the acclimation period. Animal weights were taken weekly for the study duration. Rats were randomly divided into 7 groups (n=3 per group). Group 1 (n=3) was euthanized prior to the start HFC-F diet and served as a baseline control. The remaining 6 groups (n=18) were fed a high fat and cholesterol with fructose diet for 4 weeks (HFC-F diet). Group 2 (n=3) was euthanized after 4 weeks on the HFC-F diet to monitor development of metabolic abnormalities. On the euthanasia days of groups 1 and 2, gross examination was performed, and liver weights were recorded. The blood (serum), liver and adipose tissue were collected for biochemical and histological (liver only) analysis. Samples for biochemical evaluation were stored at −80° C., and the liver sample for histology was fixed and stored in 10% Neutral Buffer Formalin (NBF).

Treatment: After 4 weeks on the HFC-F diet, the remaining 5 groups of animals were treated (n=3 per group). Group 7 served as an untreated control. Group 3, 4, 5, and 6 received one injection of 2 mL adipose formulation #1, 2, 3 and 4 in four points in the abdominal subcutaneous fat, respectively. Remaining after injections adipose formulations was fixed and stored in 10% NBF for histological analysis. Animals continued to stay on the HFC-F diet for an additional 4 weeks. Four weeks after dosing rats were euthanized, and gross examination and liver weighing were performed. The blood (serum), liver and adipose tissue were collected for biochemical and histological (liver only) analysis. The injection sites were examined, and the adipose formulations were photographed and dissected out. One half of the adipose formulation was collected in tubes with medium for cellular and biochemical testing, and the other half of the formulation was fixed in 10% NBF for histological analysis.

### Sample Analyses:

Serum samples were collected from each animal and aliquoted. Serum samples were analyzed for blood biochemistry, cytokines and oxidized lipids.

Histology: Histology of liver and adipose formulations was performed at a histology lab. Liver histology included H&E and Masson's trichrome staining and microscopic grading of stained sections. Grading was performed as described by Matsunami et al, Int J Clin Exp Pathol, 2010, 3(5):472-481. Briefly, the degree of steatosis was scored as the percentage of hepatocytes containing lipid droplets. Inflammation was evaluated as the sum of scores (score 0-6) for acinar inflammation (score 0-3) and portal inflammation (score 0-3). Fibrosis was graded from 0 (absent) to 4 (1, perisinusoidal/pericellular fibrosis; 2, periportal fibrosis; 3, bridging fibrosis; 4, cirrhosis). Adipose formulation histology included H&E staining. Five different fields per each section were examined and graded. Parameters include: the presence of necrosis; tissue integrity (adipocyte formation); inflammation (host cell infiltration); and fibrosis. Each parameter was be graded on a semiquantitative scale ranging from 0 to 4 by the relative presence of each of the histologic parameters in the slide: 0 (absence), 1 (1-25% tissue involved), 2 (26-50% tissue involved), 3 (51-75% tissue involved), 4 (76-100% involved). H&E-stained adipose formulations prior to injection will be serve as controls. Additional histological or immunohistochemical staining can be added if required.

Adipose formulation immunohistochemistry (ICH): After completion of the study, an immunohistochemical (ICH) staining of the adipose formulations was performed to detect the presence of M2 macrophages (the CD206 marker) and the expression of the CPTIA lipid oxidation enzyme. Rat subcutaneous adipose and formulation #4 prior to injection was used as controls. Antibodies: Rabbit mAb CD206 at 1:400 dilution (CellSignaling; cat #24595); rabbit polyclonal Ab CPTIA at 1:1600 dilution (ProteinTech, cat #15184-1-AP).

### Results:

Body weights. Body weights were measured weekly until the end of the study. Data shows that all rats were gaining weight as expected. Table 9 summarizes the body weight for animals from control and treated groups at the end of the study (day 56). There were no differences in body weight between control and treated groups. There was no weight gain.

TABLE 9
summarizes body weights for animals from control
and treated groups at the end of the study (Day 56):
BODY WEIGHT (g)

| | Control | Treatment Groups | | | |
|---|---|---|---|---|---|
| Individual | (8 weeks | Formu- | Formu- | Formu- | Formu- |
| animals in | on HFC-F | lation #1: | lation #2: | lation #3: | lation #4: |
| each group | diet) | M1 | M1 + cells | M2 | M2 + cells |
| 1 | 639 | 684 | 628 | 609 | 662 |
| 2 | 597 | 657 | 629 | 617 | 664 |
| 3 | 716 | 584 | 624 | 690 | 618 |
| Mean | 651 | 642 | 627 | 639 | 648 |
| SD | 60 | 52 | 3 | 45 | 26 |
| P-values for | | 0.8541 | 0.5346 | 0.7956 | 0.9473 |
| control (8 | | | | | |
| weeks) vs | | | | | |
| treatment | | | | | |
| groups | | | | | |

Blood Biochemistry. Tables 10-17 summarize the liver enzymes (Tables 10-12), lipids (Tables 13-15), glucose (Table 16) and phosphorus (Table 17) results for all study groups. Animal B30-106-006 developed severe wounds, and blood serum results for this animal were excluded from analysis. General observations for blood serum chemistry results: i) levels of all measured analytes (except cholesterol) were significantly higher the normal values for the baseline group.

A statistically significant decrease in cholesterol was recorded for rats treated with M1, M1+ cells and M2+ cells formulations. This result is in line with results of when treatment with the matrix+ cells formulation led to cholesterol decrease.

Statistically significant increase in liver enzymes AST and ALT and in oxidized lipids in animals treated with M1 and M1+ cells formulations.

M2+ cells formulation shows a statistically significant decrease in cholesterol and a positive trend to decrease in triglycerides and liver enzymes AST and ALT, but without statistical significance.

HFC-F diet triggered an increase in oxidized lipids in serum, and the treatment of animals with M2+ cells formulation prevented the increase.

ASPARTATE AMINOTRANSFERASE (AST, IU/L, normal range: 10-45 IU/L)

| Individual | Baseline | | Control | Treatment Groups | | | |
|---|---|---|---|---|---|---|---|
| animals in | prior to | 4 weeks on | (8 weeks on | Formulation #1: | Formulation #2: | Formulation #3: | Formulation #4: |
| each group | HFC-F diet | HFC-F diet | HFC-F diet) | M1 | M1 + cells | M2 | M2 + cells |
| 1 | 111 | 227 | 189 | 1114 | 1559 | 305 | 215 |
| 2 | 788 | 430 | 219 | 715 | 1174 | 153 | 77 |
| 3 | 669 | 156 | 179 | 1867 | Sick animal | 244 | 193 |
| | | | | | excluded | | |
| Mean | 523 | 271 | 196 | 1232 | 1367 | 234 | 162 |
| SD | 361.44 | 142.20 | 20.82 | 584.99 | 272.24 | 76.49 | 74.14 |
| P-values for | | | | 0.037423 | 0.003915 | 0.449412 | 0.487076 |
| control | | | | | | | |
| (8 weeks) vs | | | | | | | |
| treatment | | | | | | | |
| groups | | | | | | | |

TABLE 11
ALANINE AMINOTRANSFERASE (ALT, IU/L, normal range: 10-35 IU/L)

| Individual | Baseline | | Control | Treatment Groups | | | |
|---|---|---|---|---|---|---|---|
| animals in | (prior to | 4 weeks on | (8 weeks on | Formulation #1: | Formulation #2: | Formulation #3: | Formulation #4: |
| each group | HFC-F diet) | HFC-F diet | HFC-F diet) | M1 | M1 + cells | M2 | M2 + cells |
| 1 | 115 | 64 | 74 | 117 | 193 | 78 | 83 |
| 2 | 444 | 169 | 74 | 167 | 194 | 69 | 43 |
| 3 | 451 | 66 | 71 | 169 | Sick animal | 75 | 65 |
| | | | | | excluded | | |
| Mean | 336.67 | 99.67 | 73.00 | 151.00 | 193.50 | 74.00 | 63.67 |
| SD | 192.00 | 60.05 | 1.73 | 29.46 | 0.71 | 4.58 | 20.03 |
| P-values for | | | | 0.010201 | 0.000003 | 0.741521 | 0.466488 |
| control | | | | | | | |
| (8 weeks) vs | | | | | | | |
| treatment | | | | | | | |
| groups | | | | | | | |

TABLE 12
ALKALINE PHOSPHATASE (ALP, IU/L, normal range: 15-45 IU/L)

| Individual | Baseline | | Control | Treatment Groups | | | |
|---|---|---|---|---|---|---|---|
| animals in | (prior to | 4 weeks on | (8 weeks on | Formulation #1: | Formulation #2: | Formulation #3: | Formulation #4: |
| each group | HFC-F diet) | HFC-F diet | HFC-F diet) | M1 | M1 + cells | M2 | M2 + cells |
| 1 | 185 | 510 | 243 | 255 | 229 | 275 | 297 |
| 2 | 218 | 381 | 448 | 292 | 242 | 281 | 140 |
| 3 | 346 | 336 | 296 | 236 | Sick animal | 279 | 256 |
| | | | | | excluded | | |
| Mean | 250 | 409 | 329 | 261 | 236 | 278 | 231 |
| SD | 85.04 | 90.32 | 106.41 | 28.48 | 9.19 | 3.06 | 81.43 |
| P-values for | | | 0.377009 | 0.345201 | 0.324188 | 0.456047 | 0.273968 |
| control | | | | | | | |
| (8 weeks) vs | | | | | | | |
| treatment | | | | | | | |
| groups | | | | | | | |

CHOLESTEROL (mg/dL, normal values: <200 mg/dL))

| Individual | Baseline | | Control | Treatment Groups | | | |
|---|---|---|---|---|---|---|---|
| animals in | prior to | 4 weeks on | (8 weeks on | Formulation #1: | Formulation #2: | Formulation #3: | Formulation #4: |
| each group | HFC-F diet | HFC-F diet | HFC-F diet) | M1 | M1 + cells | M2 | M2 + cells |
| 1 | 183 | 373 | 417 | 357 | 282 | 206 | 315 |
| 2 | 113 | 314 | 434 | 288 | 236 | 395 | 270 |
| 3 | 127 | 285 | 424 | 243 | Sick animal | 345 | 275 |
| | | | | | excluded | | |
| Mean | 141 | 324 | 425 | 296 | 259 | 315 | 287 |
| SD | 37.04 | 44.84 | 8.54 | 57.42 | 32.53 | 97.93 | 24.66 |
| P-values for | | | | 0.018320 | 0.002825 | 0.125485 | 0.000782 |
| control | | | | | | | |
| (8 weeks) vs | | | | | | | |
| treatment | | | | | | | |
| groups | | | | | | | |

TRIGLYCERIDES (mg/dL, normal values: <150 mg/dL)

| Individual | Baseline | | Control | Treatment Groups | | | |
|---|---|---|---|---|---|---|---|
| animals in | (prior to | 4 weeks on | (8 weeks on | Formulation #1: | Formulation #2: | Formulation #3: | Formulation #4: |
| each group | HFC-F diet) | HFC-F diet | HFC-F diet) | M1 | M1 + cells | M2 | M2 + cells |
| 1 | 386 | 346 | 937 | 506 | 332 | 234 | 287 |
| 2 | 367 | 789 | 425 | 494 | 503 | 925 | 308 |
| 3 | 1346 | 1486 | 350 | 294 | Sick animal | 644 | 538 |
| | | | | | excluded | | |
| Mean | 700 | 874 | 571 | 431 | 418 | 601 | 378 |
| SD | 560 | 575 | 319 | 119 | 121 | 348 | 139 |
| P-values for | | | | 0.518103 | 0.578367 | 0.916737 | 0.391761 |
| control | | | | | | | |
| (8 weeks) vs | | | | | | | |
| treatment | | | | | | | |
| groups | | | | | | | |

OXIDIZED LIPIDS (μM)

| Individual | Baseline | | Control | Treatment Groups | | | |
|---|---|---|---|---|---|---|---|
| animals in | (prior to | 4 weeks on | (8 weeks on | Formulation #1: | Formulation #2: | Formulation #3: | Formulation #4: |
| each group | HFC-F diet) | HFC-F diet | HFC-F diet) | M1 | M1 + cells | M2 | M2 + cells |
| 1 | 0.250 | 1.435 | 0.803 | 1.224 | 1.014 | 1.303 | 1.119 |
| 2 | 0.276 | 0.382 | 0.724 | 0.671 | 1.014 | 0.987 | 0.382 |
| 3 | 0.619 | 0.724 | 1.119 | 1.251 | 0.750 | 0.276 | 0.803 |
| Mean | 0.382 | 0.847 | 0.882 | 1.049 | 0.926 | 0.856 | 0.768 |
| SD | 0.206 | 0.537 | 0.209 | 0.327 | 0.152 | 0.526 | 0.370 |
| T-test (vs | | 0.234 | 0.042* | 0.040 | 0.021 | 0.220 | 0.189 |
| baseline) | | | | | | | |

GLUCOSE (mg/dL normal range: 60-125 mg/dL)

| Individual | Baseline | | Control | Treatment Groups | | | |
|---|---|---|---|---|---|---|---|
| animals in | prior to | 4 weeks on | (8 weeks on | Formulation #1: | Formulation #2: | Formulation #3: | Formulation #4: |
| each group | HFC-F diet | HFC-F diet | HFC-F diet) | M1 | M1 + cells | M2 | M2 + cells |
| 1 | 139 | 98 | 104 | 210 | 230 | 147 | 130 |
| 2 | 181 | 105 | 100 | 151 | 253 | 113 | 113 |
| 3 | 218 | 153 | 105 | 206 | Sick animal | 116 | 120 |
| | | | | | excluded | | |
| Mean | 179 | 119 | 103 | 189 | 242 | 125 | 121 |
| SD | 39.53 | 29.94 | 2.65 | 32.97 | 16.26 | 18.82 | 8.54 |
| P-values for | | | | 0.010794 | 0.000557 | 0.111579 | 0.025221 |
| control | | | | | | | |
| (8 weeks) vs | | | | | | | |
| treatment | | | | | | | |
| groups | | | | | | | |

PHOSPHORUS (mg/dL, normal range: 4.2-8.5 mg/dL)

| Individual | Baseline | | Control | Treatment Groups | | | |
|---|---|---|---|---|---|---|---|
| animals in | (prior to | 4 weeks on | 18 weeks on | Formulation #1: | Formulation #2: | Formulation #3: | Formulation #4: |
| each group | HFC-F diet) | HFC-F diet | HFC-F diet) | M1 | M1 + cells | M2 | M2 + cells |
| 1 | 10.7 | 9.80 | 7.70 | 11.60 | 13.20 | 7.60 | 8.00 |
| 2 | 12.4 | 8.80 | 7.20 | 7.80 | 10.30 | 7.60 | 8.50 |
| 3 | 10.9 | 9.30 | 7.60 | 13.00 | Sick animal | 7.60 | 7.70 |
| | | | | | excluded | | |
| Mean | 11.33 | 9.30 | 7.50 | 10.80 | 11.75 | 7.60 | 8.07 |
| SD | 0.93 | 0.50 | 0.26 | 2.69 | 2.05 | 0.00 | 0.40 |
| P-values for | | | | 0.102023 | 0.030558 | 0.548424 | 0.111972 |
| control | | | | | | | |
| (8 weeks) vs | | | | | | | |
| treatment | | | | | | | |
| groups | | | | | | | |

Tissue collection. Tissues were collected as described in the protocol. Noticeable increase in the abdominal fat over time in the rats that were fed the HFC-F diet. There were no visual differences between control and treated groups and between animals in the group (Data not shown).

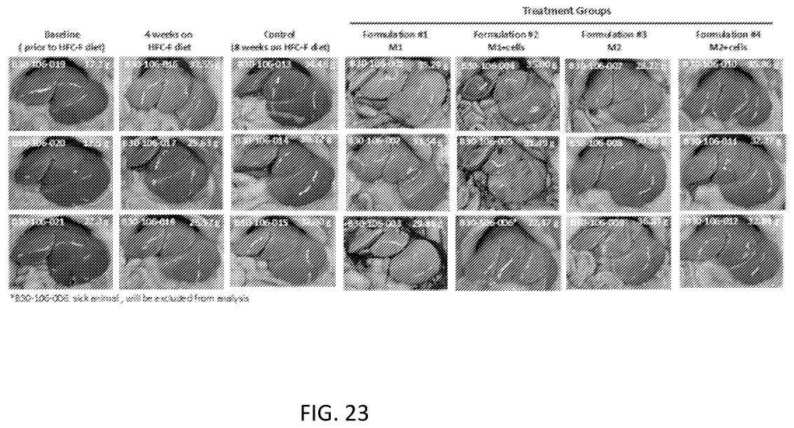

FIG. 23 shows changes in liver size and color between the baseline (prior to HFC-F diet) and all other groups. In comparison to the baseline, which shows normal liver appearance, all livers at weeks 4 and 8 were larger and pale in color (greyish), which is a sign of the fatty liver disease (FIG. 23). There were no visual differences between control and treatment groups and between animals in the group (FIG. 23). Liver weights are summarized in Table JJJ. Rat B30-103-006 developed several severe chronic wounds and was excluded from the analysis. Liver weights were statistically significant larger in all groups versus the baseline group, however, there is no statistical difference in liver weight between control 8 weeks and treatment groups (Table 18). Although liver weights were not different between four treatment groups the color of livers from animals in the M2+ cells group is less greyish, close to the color of the normal liver (FIG. 6, last set of images on the right). Collected livers were divided into portions: 3 small pieces from different liver lobes—in 1×50 mL tube with a fixative solution for histology; remaining livers—in 1×50 mL tubes. All 50 mL tubes were flash frozen in liquid nitrogen, and then transferred for storage to a −80° ° C. freezer prior to further testing. Subcutaneous abdominal adipose was also collected from each animal. Therapeutic formulations were identified and excised from the adipose. One part of the excised formulations was fixed in 10% formalin. Remaining formulations and collected adipose were flash frozen in liquid nitrogen, and then transferred for storage to a −80° C. freezer.

LIVER WEIGHT (g)

| Individual | Baseline | | Control | Treatment Groups | | | |
|---|---|---|---|---|---|---|---|
| animals in | (prior to | 4 weeks on | (8 weeks on | Formulation #1: | Formulation #2: | Formulation #3: | Formulation #4: |
| each group | HFC-F diet) | HFC-F diet | HFC-F diet) | M1 | M1 + cells | M2 | M2 + cells |
| 1 | 17.2 | 32.55 | 34.48 | 35.29 | 35 | 28.22 | 35.86 |
| 2 | 17.5 | 25.63 | 30.72 | 33.54 | 31.49 | 30.96 | 32.27 |
| 3 | 22.3 | 25.57 | 32 | 29.86 | Sick animal | 36.83 | 32.84 |
| | | | | | excluded | | |
| Mean | 19.00 | 27.92 | 32.40 | 32.90 | 33.25 | 32.00 | 33.66 |
| SD | 2.86 | 4.01 | 1.91 | 2.77 | 2.48 | 4.40 | 1.93 |
| P-values for | | | | 0.81 | 0.69 | 0.89 | 0.47 |
| control | | | | | | | |
| (8 weeks) vs | | | | | | | |
| treatment | | | | | | | |
| groups | | | | | | | |
| P-values for | | 0.04 | 0.0025 | 0.0038 | 0.0107 | 0.0128 | 0.0018 |
| baseline | | | | | | | |
| vs other groups | | | | | | | |

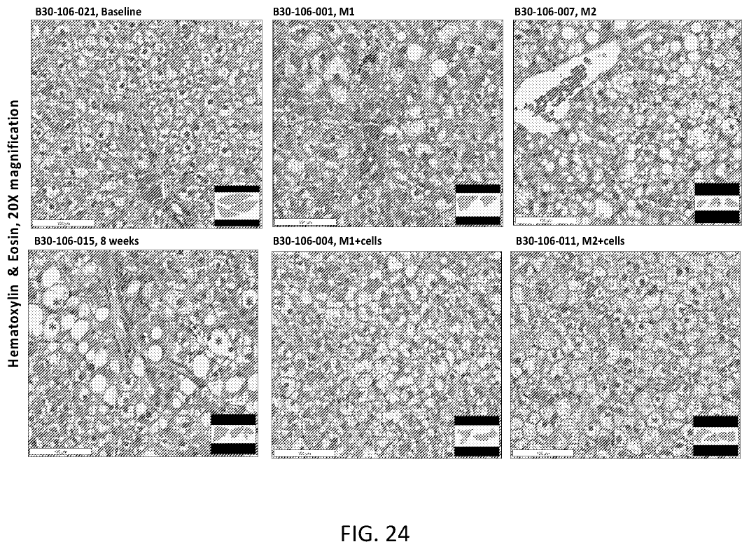

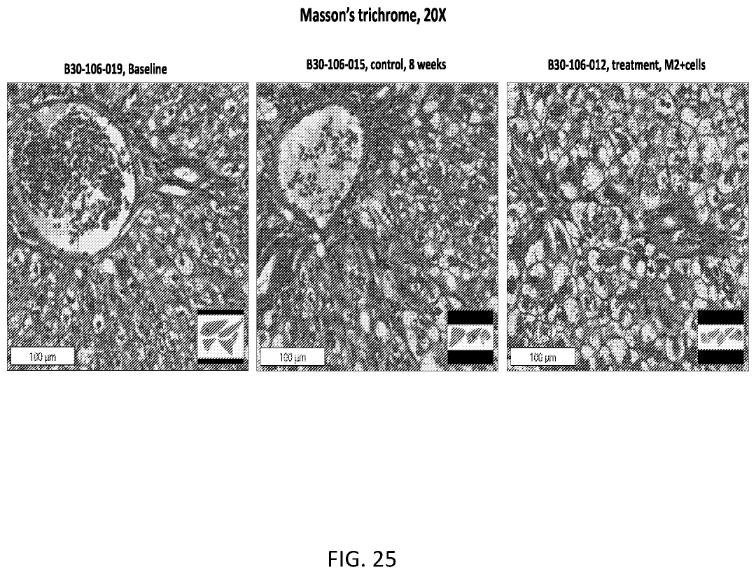

Histological analysis. Collected liver tissues and four therapeutic formulations were fixed in 10% NBF and sent to a histology lab for H&E and MT staining. Stained sections were analyzed by a certified pathologist. All animals from all groups on HFC-F diet developed liver steatosis (FIG. 24). Steatosis in three animals from 3 different groups was scored “3”, and all other animals developed score “4” (maximal score) steatosis. Inflammation was not present in the liver, and minimal liver fibrosis was detected in 2 control animals: one from 4 weeks and another from 8 weeks on the HFC-F diet There was no liver fibrosis in any of treated animals. FIG. 23 shows representative images of Masson's trichrome stained liver sections from the baseline, 8 weeks control and M2+ cells treated animals. The liver steatosis (lipid accumulation) scores are not different between the control and treatment groups, and between treatment groups. However, the H&E-stained liver sections show that M1+ cells and M2+ cells treated animals developed predominantly microvesicular steatosis and had smaller size of hepatocytes. In contrast, control animals developed macrosteatosis (FIG. 25). Animals treated with formulations without cells also developed macrovesicular steatosis (FIG. 25). Microvesicular steatosis is intermediate to macrovesicular steatosis (Kristiansin et al. Lipids, 2019; 54:109), which is a positive outcome of the treatment with formulations containing cells. Analysis of liver triglyceride content shows statistically significant increase triggered the by HFC-F diet (Table 19). Treatment of animals with the M2+ cells formulation shows a trend to reduction in liver triglycerides, but the effect did not reach statistical significance. Other formulations showed no effect.

TRIGLYCERIDES (mg/mg liver tissue)

| Individual | Baseline | | Control | | | | |
|---|---|---|---|---|---|---|---|
| animals in | (prior to | 4 weeks on | (8 weeks on | Treatment at 8 weeks | | | |
| each group | HFC-F diet) | HCF-F diet | HFC-F diet) | M1 | M1 + cells | M2 | M2 + cells |
| 1 | 0.0369 | 0.063991 | 0.045086 | 0.0468 | 0.047231 | 0.0651 | 0.0462791 |
| 2 | 0.02902 | 0.046946 | 0.066329 | 0.0811 | 0.061469 | 0.0533 | 0.0435298 |
| 3 | 0.03722 | 0.064471 | 0.05188 | 0.0855 | Sick | 0.0662 | 0.0445824 |
| | | | | | animal | | |
| Mean | 0.03438 | 0.058469 | 0.054432 | 0.0711 | 0.05435 | 0.0616 | 0.0447971 |
| SD | 0.00465 | 0.009982 | 0.010849 | 0.0212 | 0.010068 | 0.0072 | 0.0013871 |
| T-test | | 0.019269 | 0.042255 | 0.2913 | 0.993814 | 0.3954 | 0.2017742 |

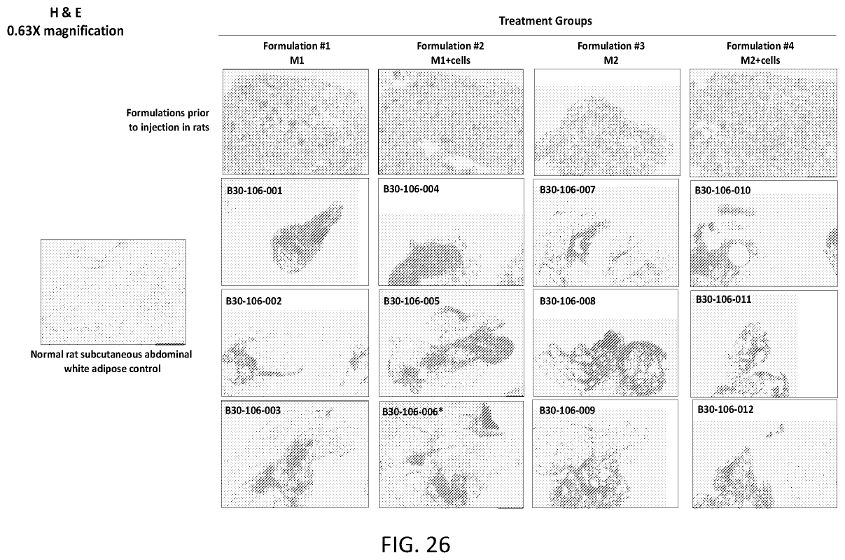

FIG. 26 shows histological appearance of the excised formulations 4 weeks post-injection in rats at a low magnification. The formulations prior to injections and normal rat white adipose sections are included in FIG. 26 for comparison. Table MMM below summarizes histological scores.

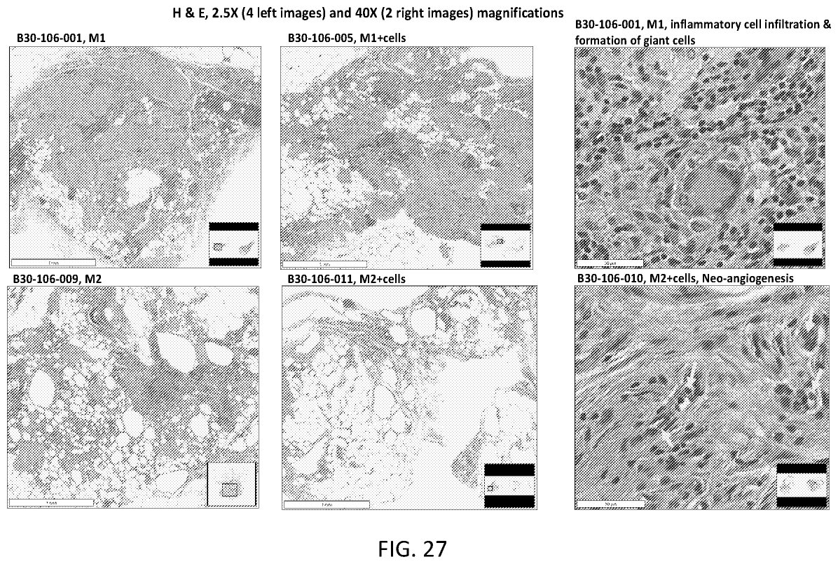

The M1 formulation has highest scores for inflammation and for the presence of giant cells and the lowest score for angiogenesis (new blood vessel formation). The presence of giant cells (multinuclear cells composed of fused macrophages) indicates a “foreign body” response. The M2+ cells formulation has lowest scores for inflammation and giant cells and highest score for angiogenesis. FIG. 27 illustrates scoring in Table MMM. Four representative H&E images for each formulation at a higher magnification are shown on the left side of FIG. 27. Formulations containing the M1 matrix with and without cells show high number of inflammatory cells infiltrating implants. Two images on the right side of FIG. 27 show images of inflammatory cells and giant cells in the M1 formulation (the upper image) and the presence of newly formed blood vessels (neo-angiogenesis) in M2+ cells formulation (the bottom image).

TABLE 20
summarizes liver histological scores for four
therapeutic formulations 4 weeks post-injection.
Histological Scores (mean +/− SD)

| | Formulation #1: | Formulation #2: | Formulation #3: | Formulation #4: |
|---|---|---|---|---|
| | M1 | M1 + cells | M2 | M2 + cells |
| Collagen matrix in the | 4.00 | 4.00 | 3.00 | 4.00 |
| formulation prior to | | | | |
| implantation | | | | |
| Collagen matrix | 2.67 +/− 0.58 | 3.33 +/− 0.58 | 3.00 +/− 0.00 | 3.00 +/− 0.00 |
| Inflammation | 2.67 +/− 0.58 | 2.33 +/− 0.58 | 2.33 +/− 0.58 | 2.00 +/− 0.00 |
| Giant cells | 2.67 +/− 0.58 | 2.33 +/− 0.58 | 2.00 +/− 0.00 | 1.33 +/− 0.58 |
| Angiogenesis | 0.67 +/− 1.15 | 0.33 +/− 0.58 | 1.00 +/− 1.00 | 1.00 +/− 1.00 |
| Adipose | 2.67 +/− 0.58 | 2.67 +/− 0.58 | 2.00 +/− 0.00 | 2.00 +/− 0.00 |

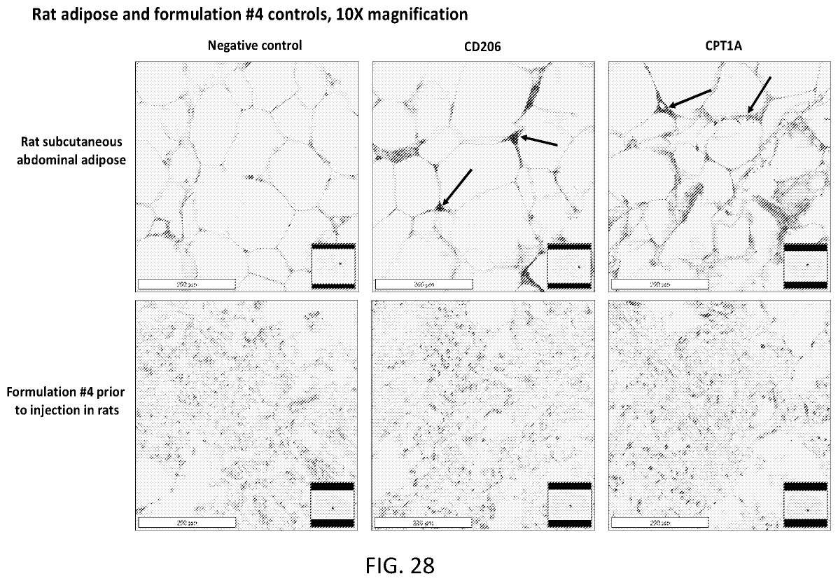

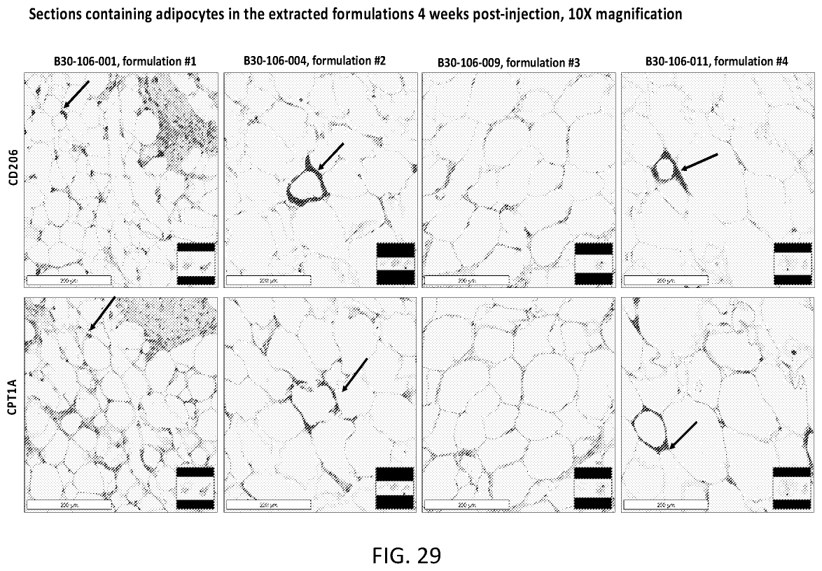

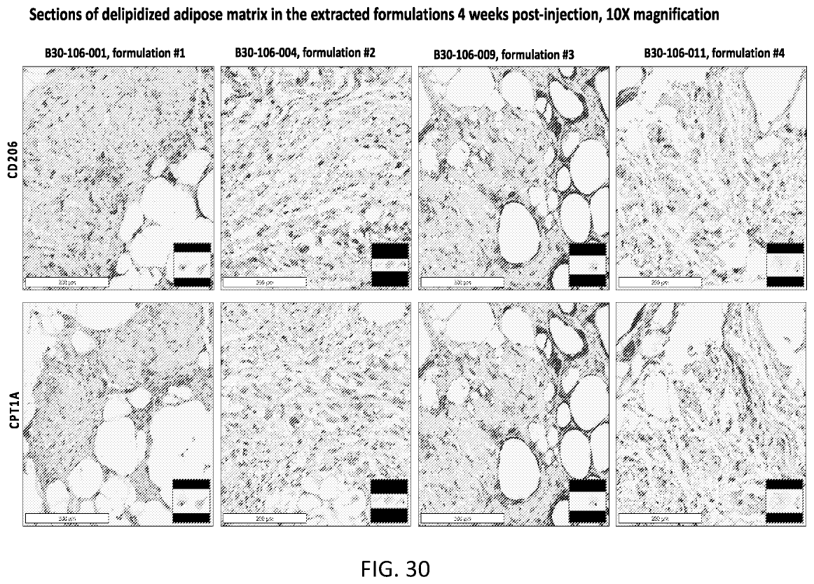

Immunohistochemical analysis. An immunohistochemical (ICH) staining of the adipose formulations was performed to detect the presence of M2 macrophages (the CD206 marker) and the expression of the CPTIA lipid oxidation enzyme. Rat subcutaneous adipose and formulation #4 prior to injection were used as controls. Antibodies were titrated using rat subcutaneous adipose tissue from the baseline animal B30-106-019 and formulation #4 to determine the optimal dilution for ICH. Rabbit mAb CD206 were used at 1:400 dilution (Cell Signaling; cat #24595), and rabbit polyclonal Ab CPTIA were used at 1:1600 dilution (Proteintech, cat #15184-1-AP). ICH staining was performed for formulation #1 from rat B30-106-001; formulation #2 from rat B30-106-004, formulation #3 from rat B30-106-009 and formulation #4 from rat B30-106-011. FIG. 28 shows that the adipose formulations prior to injection had neither CD206 nor CPTIA positive cells. However, 4 weeks after the injection these formulations were infiltrated by host CD206 cells (M2 macrophages), which are expressing the CPTIA enzyme (FIGS. 29 and 30). Also, clusters of giant cells that CPTIA positive, but CD206 negative were detected in the formulations. FIG. 28 shows such clusters in formulation #4.

### Summary

The purpose of this study was to evaluate the therapeutic potential of four adipose formulations to treat NASH and metabolic abnormalities in obese fa/fa Zucker rats. The study was conducted using 21 male 8-10 weeks old obese fa/fa Zucker rats. The duration of the study was 9 weeks including the acclimation period. Animal weights were taken weekly for the study duration. Rats were randomly divided into 7 groups (n=3 per group): baseline (prior to the start HFC-F diet), 4 weeks control (animals were fed the HFC-F diet for 4 weeks), untreated control and 4 treatment groups. The treatment of animals was performed after 4 weeks on the HFC-F diet. Animals were divided in 4 treatment groups, and each animal received one injection of 2 mL adipose formulation #1, 2, 3 or 4 in four points in the abdominal subcutaneous fat. Animals continued to stay on the HFC-F diet for an additional 4 weeks.

The HFC-F diet triggered severe liver steatosis. Fibrosis (score 1) was detected in one untreated control and one 4 weeks on HFC-F diet control animals. Development of liver steatosis correlated with the increase in blood serum lipids.

### Results Demonstrate:

The effect on liver steatosis was limited to micro- vs macrosteatosis for cell-containing formulations #2 and #4 vs matrix only formulations #1 and #3.

There were statistically significant decreases in cholesterol with the strongest effect for formulation #4 (partially delipidized adipose matrix+ cells).

Treatment with formulation #4 prevented HFC-F-induced lipid oxidation in serum.

Treatment with formulations #1 and #2 resulted in high inflammatory response to the implants determined histologically by high number of inflammatory cells infiltrating these implants and by formation of high number of giant cells.

Blood biochemistry analysis shows high levels of enzymes AST and ALT after treatment with formulations #1 and #2, which an indicator of inflammation and/or tissue damage.

Exploratory ICH staining of formulations post-injection demonstrates that all implants were infiltrated by M2 macrophages, which are positive for the CPT1A lipid oxidation enzyme.

In conclusion, formulation #4, which was comprised of a partially delipidized adipose matrix combined with adipose-derived MSCs, exhibited a good safety profile and positive effects on liver steatosis and serum lipids.

### F. Example 6 In Vitro Tissue Immunogenicity Testing

A LPS challenge assay was used to assess tissue immunogenicity. Pieces of tissues or cells isolated from collagenase digested tissue (SVF and adipocytes) were incubated in 24-well plates in the presence of 10 μg/mL LPS for tissues and 1 μg/mL LPS for isolated cells for 48 h. Wells without LPS were served as a control. After 48 h tissue culture supernatants were collected and tested for the presence of TNF-α using a high-sensitive human TNF Luminex kit (Thermofisher) according to the manufacturer's protocol.

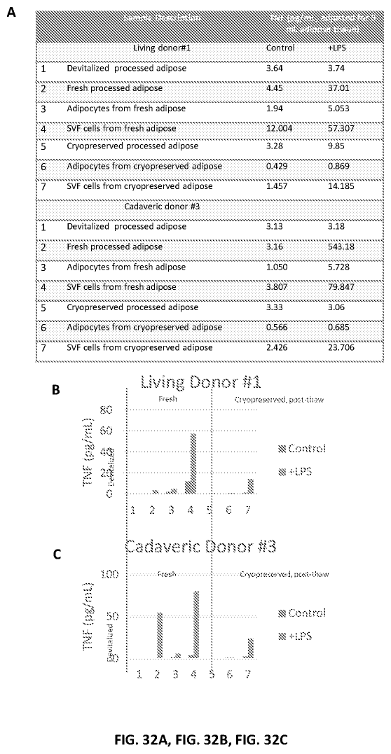

As a part of the assay development fresh adipose tissue prior to and after processing, adipocytes and SVF cells isolated from fresh adipose after the processing, cryopreserved processed adipose and adipocytes and SVF cells isolated from cryopreserved adipose were tested. Devitalized tissue served as a negative control. Results presented in FIG. 32 show that the fresh processed adipose tissue or isolated SVF cells release the highest levels of TNF-α upon LPS stimulation, and that cryopreservation significantly reduces levels of TNF-α. SVF cells isolated from each donor were tested in the LPS challenge assay. For all donors TNF-α levels released from SVF cells after stimulation with LPS were approximately 50 or less pg/mL, which corresponds to 5 mL of the processed viable cryopreserved human adipose tissue product.

### G. Example 7: Evaluation of Human Cryopreserved Devitalized and Viable Adipose Tissues in an Immune Competent Mouse Model

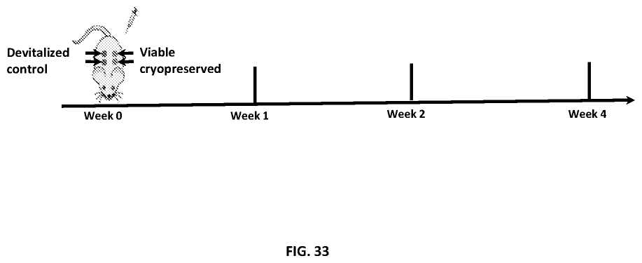

Study design: This study was conducted using ten (10), eight to ten weeks (8-10) weeks old female FVB.129P2-Pde60/AntJ mice. The duration of the study was 4 weeks. After undergoing general anesthesia, 10 mice received subcutaneous injections of ˜0.25-0.3 mL/point of devitalized and viable cryopreserved human adipose grafts. The adipose grafts were injected via a 18 G needle paravertebrally at 2 points on the left side for devitalized cryopreserved graft, and at 2 points on the right side for viable cryopreserved graft. Each mouse received a total of 4 injections of human adipose. Body weights were taken weekly. Mice (n=3) from each experimental group were sacrificed at weeks 1, 2 and 4 post-injection. FIG. 33 shows the study design.

Sample collection: The skin on the back of each mouse was cut, and the adipose grafts were photographed and dissected out. One devitalized cryopreserved graft and one viable cryopreserved graft from each animal was fixed in formalin for histological analysis, and one devitalized cryopreserved graft and one viable cryopreserved graft from each animal was snap frozen on dry ice for cytokine and gene expression analyses. Two pieces of mouse adipose tissue were collected from one animal and fixed and snap frozen to be used as a reference control for histology and cytokines and gene expression, respectively. Fixed samples were sent to a histology lab, and frozen samples were stored at −80 C prior to testing.

Histological evaluation: fixed samples were stained by hematoxylin and eosin (H&E) using a standard protocol at a certified histology lab. Histological analysis included evaluation of tissue inflammation and morphology of H&E stained devitalized and viable cryopreserved human adipose grafts collected from the same animal. Five different fields per each graft type per each animal and time point were evaluated and graded. The parameters included: the presence of cysts, vacuoles and necrotic nodules; inflammation, as evidenced by cell infiltration of the grafts; and the presence of fibrosis and other components of the connective tissue (i.e., collagen and elastic fibrils). Each of these parameters was graded on a semiquantitative scale ranging from 0 to 4 by evaluation of the relative presence of each of the histologic parameters in the slide under examination, as follows: 0 (absence), 1 (minimal presence, ˜25%), 3 (moderate presence, ˜50%), 4 (extensive presence 75%). Mean semiquantitative scores for 5 fields of the lyophilized grafts were compared to the score for the cryopreserved grafts at each time point. The presence of fibrosis and other components of the connective tissue (i.e., collagen and elastic fibrils) was confirmed by Masson's trichrome staining of devitalized and viable cryopreserved human adipose grafts at week 4. Immunohistochemical staining of devitalized and viable cryopreserved human adipose grafts at weeks 4 with mouse F4/80, mouse CD3 and human KU80 was performed to detect infiltration of grafts with mouse macrophages and T-cells, and to detect the presence of human cells in grafts, respectively. Histological staining was performed at a certified histology lab, and all samples were evaluated by a certified independent pathologist.

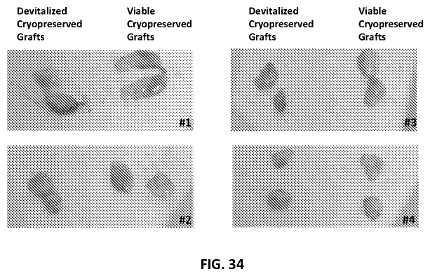

FIG. 34 shows visual appearance of devitalized (the left side on each photograph) versus viable (the right side on each photograph) cryopreserved human adipose grafts excised from fully immunocompetent mice 4 weeks after implantation. All grafts were present in mice after 4 weeks without significant differences between grafts.

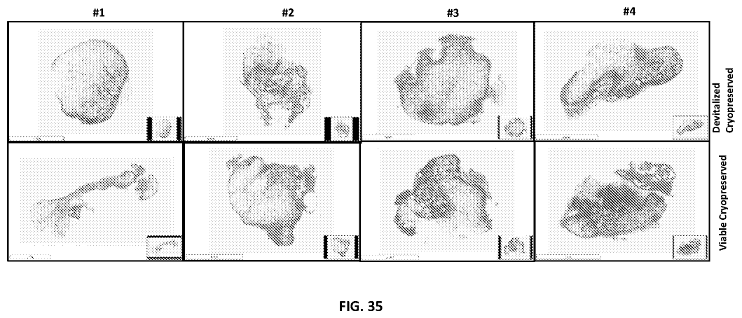

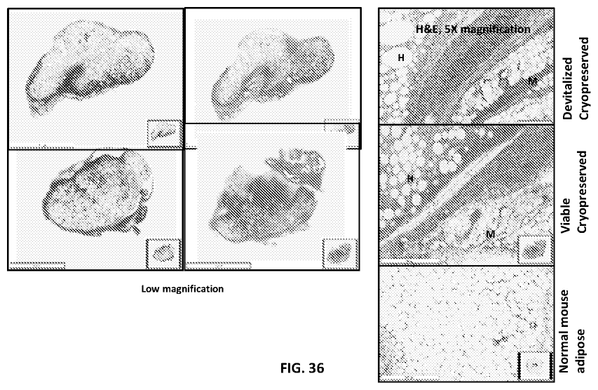

The structure of the excised grafts after 4 weeks post-implantation was evaluated histologically using H&E stained sections. FIG. 35 shows H&E stained graft section at low magnification. H&E staining shows that there are no significant differences between devitalized vs viable cryopreserved grafts. The lack of significant differences was confirmed by histological scores (Table 21). It was an unexpected finding, particularly for inflammation scores: although the inflammation score was higher (=more inflammatory response against human antigens) for the viable cryopreserved grafts the difference was not statistically significant (Table 21). Another unexpected finding is that the mechanism of tissue remodeling is similar for both devitalized (no living human cell in the graft) and viable human cryopreserved adipose grafts. Masson's trichrome (MT) staining shows the same amount and pattern of collagen deposition in both grafts (FIG. 36, two images on the left), and in both grafts there are areas representing human adipose tissue necrosis and regenerated new adipose tissue (FIG. 36, images on the right), which are consistent with the appearance of normal mouse adipose (FIG. 36, the bottom image on the right).

TABLE 21
shows mean histological scores for devitalized versus
viable cryopreserved human adipose grafts excised from
fully immunocompetent mice 4 weeks after implantation

WEEK 4, Histological Scores (Mean ± SD)

| | Devitalized | Viable | |
|---|---|---|---|
| Evaluation | Cryopreserved | Cryopreserved | |
| Parameters | Graft | Graft | P |
| Integrity | 2.25 ± 0.50 | 2.00 ± 1.15 | 0.761 |
| Oil Vacuoles | 2.00 ± 0.00 | 2.00 ± 1.15 | 1.00 |
| Fibrosis | 2.75 ± 0.50 | 2.00 ± 0.82 | 0.058 |
| Inflammation | 1.00 ± 0.00 | 1.5 ± 0.58 | 0.182 |

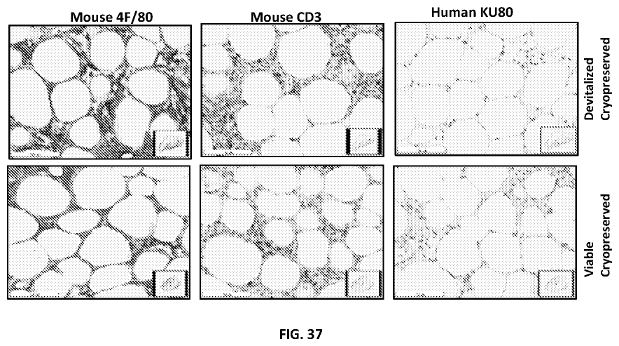

The grafts excised from mice after 4 weeks post-implantation were further evaluated using immunohistochemical staining. The staining provided answers to two important questions: 1. To identify types of immune cells that infiltrated the grafts; and 2. To detect the human cells in the grafts. FIG. 37 summarizes the results. In both grafts the major type of mouse immune cells infiltrating the grafts are macrophages (FIG. 37, left images stained for 4F/80 mouse macrophages marker). Mouse T-cells are also infiltrated the grafts (FIG. 37, middle images), however, the amount of T-cells is significantly lower as compared to the amount of macrophages. There were no differences between human adipose grafts in the level of infiltration by macrophages and T-cells. This result indicates that the inflammatory and immune responses to the graft material is mediated by human antigens rather than by the presence of living human cells in the viable cryopreserved human adipose graft.

Staining of human adipose grafts with human nuclear KU80 antigen shows that there are no human cells detectable in both grafts 4 weeks after the implantation (FIG. 37, images on the right).

In summary, this study demonstrates that there were no differences between devitalized vs vitalized and allogeneic adipose. Results supports that the developed process selectively reduces immunogenicity of adipose tissue allowing its allogeneic use.

### H. Example 8—Testing of MSC Formulations in a Delayed Healing Ischemic Wound Model in Obese fa/fa Zucker Rats Fed the High Fat, Cholesterol and Fructose (HFC-F) Diet

Restoration of adipose functionality together with anti-inflammatory and angiogenic properties of the MSCs seeded on devitalized, partially delipidized adipose matrix (MSC formulation) indicate that this therapeutic formulation might be effective in treating of ischemic wounds. A decrease in adipose functionality due to aging or metabolic diseases (one example is type 2 diabetes) is linked to impaired angiogenesis in the injured skin, the root cause of tissue ischemia in the wound. One hypothesis is that when injected in the wound area, the MSC formulation can replace dysfunctional subcutaneous adipose in the wound leading to formation of new microvasculature and re-initiation of wound healing. Regenerated adipose can also provide mechanical support required for durable closure and prevention of wound recurrence. To test the hypothesis an ischemic wound model in obese Zucker rats was selected. This model satisfies all criteria required for evaluation of the MSC formulation: obese Zucker rats have dysfunctional adipose, tissue ischemia and impaired wound healing.

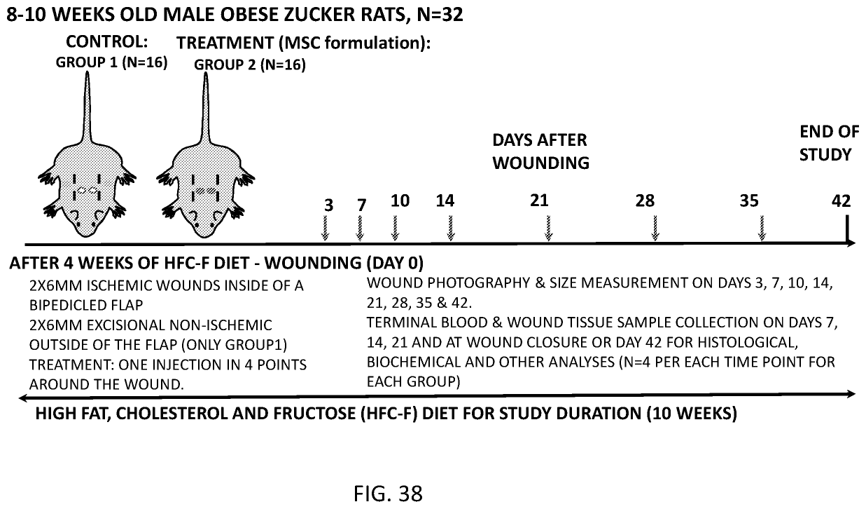

Design of Study (FIG. 38): the aim of the study is to evaluate effectiveness of the MSC formulation (human and rat-derived) in an impaired ischemic wound model in obese Zucker rats fed a HFC-F diet. This study can be conducted using 32 (n=16 per group, n=4 per each tissue collection time point) male 8-10 weeks old obese Zucker rats. The duration of the study will be 10 weeks after the animal acclimation time. All animals are fed the HFC-F diet for 4 weeks. After 4 weeks on the HFC-F diet rats can be randomly divided into 2 groups: control (n=16) and the MSC formulation treatment group (n=16). First, under isofluorane anesthesia 2×6 mm excisional full-thickness ischemic wounds can be created within a bipedicled flap on the back of each animal (n=32) as described by Trujillo et al (the copy of this paper is attached). In all animals (n=16) from the control group 2×6 mm excisional full-thickness non-ischemic wounds (control wounds) can be created outside of the bipedicled flap. The presence of ischemia inside the bipedicled flap will be confirmed by laser Doppler. Then, all animals assigned to the BRC001 treatment group (n=16, 32 wounds) will receive one subcutaneous injection of 0.2 mL of human adipose formulation in perivascular area of each wound. The MSC formulation injection can be performed into 4 points (˜50 μL per each point) of subcutis at the wound periphery. PBS can be injected around each ischemic wound in the control group (n=16, 32 wounds). Wounds can be covered by transparent sterile dressings. Wounds can be photographed at days 0, 3, 7, 10, 14, and then once weekly until complete closure or at day 42 post-treatment. All animals can continue to stay on the HFC-F diet prior to euthanasia. All animals can be weighed weekly for the study duration. Euthanasia can be performed on days 7, 14, 21 and 42 post-wounding. On euthanasia days, gross examination of animals can be performed. The blood serum and wound tissue samples can be collected from each animal. Tissue from one wound can be collected in tubes with medium for biochemical and cellular analyses, and tissue from the second wound can be fixed in 10% formalin for histological and immunohistochemical (ICH) analyses. Sample analyses can include: i) Histological and ICH analyses of wound tissue sections: H&E and Masson's trichrome staining and ICH for CD206 and CD31, and KU-80 (marker for human cells); ii) Blood serum biochemistry panel; iii) Wound tissue biochemical analyses will include growth factor and cytokine profiles, antioxidants superoxide dismutase and glutathione: iv) Wound tissue cellular analyses will include detection of human cells among cells isolated from digested tissues; v) Rate of wound healing: the ImageJ software will be used to measure the wound area and calculate the wound healing rate at each time point: the wound healing rate=(initial wound area−time point area)/initial wound area×100%; vi) Histological assessment of wound healing: Assessment of wound healing will be performed by an independent blinded animal pathologist. Tissue regenerated in the wound area will be assessed semiquantitative using histological scores on a scale of 1-4 (1—poor, 4—best). Separate scores will be calculated for epithelial migration, granulation tissue thickness, blood vessel density. Total score will be determined as a sum of separate scores.

Those skilled in the art will recognize, or be able to ascertain using no more than routine experimentation, many equivalents to the specific embodiments of the method and compositions described herein. Such equivalents are intended to be encompassed by the following claims.

1. An adipose matrix comprising devitalized and partially delipidized adipose tissue.

2. The adipose matrix of claim 1, wherein the partially delipidized adipose tissue comprises at least 50% of the original lipids.

3. The adipose matrix of claim 1, wherein the devitalized and partially delipidized adipose tissue is minced.

4. The adipose matrix of claim 3, wherein the minced adipose tissue comprises pieces of adipose tissue less than 1 mm in size.

5. The adipose matrix of claim 1, wherein the adipose matrix comprises about 0.25-20 μg DNA per mg of adipose matrix.

6. The adipose matrix of claim 1, wherein the adipose matrix is cryopreserved or has been previously cryopreserved.

7. The adipose matrix of claim 1, wherein the adipose matrix is lyophilized or has been previously cryopreserved.

8. A composition comprising the adipose matrix of claim 1.

9. The composition of claim 8, wherein the partially delipidized adipose tissue comprises 20-70% of the original lipids.

10. (canceled)

11. (canceled)

12. The composition of claim 8, wherein the composition further comprises mesenchymal stem cells (MSCs).

13. The composition of claim 8, wherein the MSCs are adipogenic differentiated MSCs.

14. The composition of claim 8, wherein the MSCs are allogeneic to the adipose matrix or autologous to the adipose matrix.

15.-25. (canceled)

26. A method of making the adipose matrix of claim 1 comprising:
a) micronizing adipose tissue;
b) emulsifying the adipose tissue;
c) centrifuging, after emulsifying, the adipose tissue to produce two distinct layers, a top layer and a layer directly underneath the top layer, wherein free lipids are present in the top layer and partially delipidized adipose tissue is present in the layer directly underneath the top layer;
d) removing the top layer; and
e) freezing the adipose tissue before step b), before step c), before step d), before step e), or after step e), thus producing a devitalized adipose tissue, wherein steps a)-f) result in a composition comprising devitalized and partially delipidized adipose tissue.

27.-38. (canceled)

39. A method of treating a subject having a metabolic liver disease comprising administering to the subject the composition of claim 8.

40. (canceled)

41. The method of claim 39, wherein the adipose matrix is derived from the adipose of the subject who is being administered the composition, from the adipose of a subject different from the subject who is being administered the composition or from a cadaver.

42. (canceled)

43. (canceled)

44. The method of claim 39, wherein the liver disease is fatty liver disease or Non-Alcoholic SteatoHepatitis (NASH).

45.-47. (canceled)

48. A method of decreasing lipids in the liver of a subject comprising administering to the subject the composition of claim 8.

49.-57. (canceled)

58. A method of decreasing cholesterol in a subject comprising administering to the subject the composition of claim 8.

59.-67. (canceled)

68. A method of treating a subject having a metabolic disease comprising administering to the subject the composition of claim 8.

69. The method of claim 68, wherein the metabolic disease is dyslipidemia/hyperlipidemia, metabolic syndrome, obesity, type 2 diabetes, or lipodystrophy.
